(19) United States
(12) Patent Application Publication
(10) Pub. No.: US 2025/0387395 A1
(43) Pub. Date: Dec. 25, 2025

(54) TRANSDERMAL ADMINISTRATION SYSTEM FOR RISPERIDONE, AND PREPARATION METHOD THEREFOR AND USE THEREOF

(71) Applicant: NOVASTAGE PHARMACEUTICALS (SHENZHEN), LTD., Shenzhen, Guangdong (CN)

(72) Inventor: Jiansheng TANG, Shenzhen, Guangdong (CN)

(21) Appl. No.: 18/876,588

(22) PCT Filed: Jan. 3, 2024
(86) PCT No.: PCT/CN2024/070458

§ 371 Date: Dec. 18, 2024

(30) Foreign Application Priority Data
Jan. 4, 2023 (CN) 202310010005.1

(51) Int. Cl. A61K 31/519; A61K 9/70; A61K 47/10; A61K 47/12; A61K 47/14

(52) U.S. Cl. CPC A61K 31/519; A61K 9/7061; A61K 47/10; A61K 47/12; A61K 47/14

(57) ABSTRACT

The present invention relates to a risperidone transdermal administration system. More specifically, the present invention relates to a transdermal administration system for continuously delivering risperidone or a pharmaceutically acceptable salt thereof at a therapeutically effective amount of blood drug concentration within 24 hours to 14 days, and a preparation method therefor and a use thereof.

### TECHNICAL FIELD

The present invention relates to a risperidone transdermal administration system. More specifically, the present invention relates to a transdermal administration system for continuously delivering risperidone or a pharmaceutically acceptable salt thereof at a therapeutically effective amount of blood drug concentration within 24 hours to 14 days, and a preparation method therefor and a use thereof.

### BACKGROUND ART

The transdermal route of administration is a route of administration that is superior to the oral route of administration, and maintains drug concentration in the blood at a constant level by continuously delivering drugs to the systemic blood system. The transdermal route of administration not only reduces the peak-to-trough fluctuations in drug concentration in the blood, but also avoids the first-pass effect. In addition, since the transdermal route of administration avoids direct contact between drugs and excipients and the gastrointestinal system, side effects such as nausea and vomiting that are often associated with the oral route of administration are significantly reduced or eliminated. Another advantage of the transdermal route of administration is that it is not affected by diet. Administration can be easily terminated when necessary, for example, when a several side effect occurs, by removing the transdermal patch from the skin. Furthermore, transdermal patches improve patient compliance by reducing the frequency of administration. In other words, patients may easily forget to take a tablet or capsule but may not forget to apply a transdermal patch. This is especially important for elderly patients and pediatric patients. For example, if the patch is designed to be worn 24 hours a day, the patient may easily remember to change the patch at 8 a.m. every morning. For example, if a patch is designed to be worn for 3.5 days, the patient may easily remember to change the patch every Monday at 8 a.m. and every Thursday 8 p.m. For example, if the patch is designed to be worn for 7 days, the patient may easily remember to change the patch every Monday morning at 8 a.m. Transdermal patches are also clearly superior to injectables because application of transdermal patches is painless, whereas application of injectables is painful. Additionally, long acting injectable medications can only be used in hospitals.

Common dosage forms of transdermal route of administration include transdermal patch preparation. Currently common transdermal patch preparations include, but are not limited to, drug reservoir-type patches and matrix-type patches. Drug reservoir-type patch preparations are those in which the drug is contained in a reservoir with a drug-permeable base surface, and matrix-type patch formulations are those in which the drug is dissolved or dispersed in a polymeric matrix layer. Both types of designs also typically include a backing layer and a release film layer that is removed before use. In addition, patches often contain a penetration enhancer layer and an adhesive layer.

In recent years, the advantages of transdermal administration have enabled many drugs to be effectively administered via the transdermal route. These advances include the development of many physical methods to increase skin permeability and facilitate transdermal administration, e.g., using iontophoresis, electroporation, ultrasound, or microneedle. However, drugs that can be effectively and safely applied through the skin for 7 days or more without causing skin adhesion, skin irritation or sensitization are still limited.

The present invention relates to risperidone, the general formula of which is 3-[2-[4-{6-fluoro-1,2-benzisoxazol-3-yl)-1-piperidyl]ethyl]-6,7,8,9-tetrahydro-2-methyl-4H-pyrido[1,2-a]pyrimidin-4-one. The preparation method and pharmacological activity of this drug are described in U.S. Pat. No. 4,804,663 (corresponding to EP-0,196,132 (1984)).

This drug is currently widely used clinically as an antischizophrenia drug. Risperidone treats or prevents schizophrenia, mania, and dementia. Risperidone is a selective monoaminergic antagonist with unique properties. It has a high affinity for serotonergic 5-HT2 receptors and dopaminergic D2 receptors, and also binds to α1-adrenergic receptors. It has a lower affinity for H1 histaminergic receptors and α-2 adrenergic receptors, and can not bind to cholinergic receptors. In addition, risperidone treats or prevents the positive or negative symptoms of schizophrenia. Risperidone treats or prevents the positive or negative symptoms of schizophrenia, including hallucinations, delusions, and emotional withdrawal and blunted affect. In addition, it is characterized by relatively fewer extrapyramidal side effects (cold, stiffness, etc.) than traditional typical antipsychotics. Therefore risperidone is considered a very effective antischizophrenia drug that can greatly improve the quality of life of patients.

Risperidone is administered to patients orally as tablets, fine granules, and liquid preparations. However, oral administration has some disadvantages, such as susceptibility to first-pass effects in the liver after drug absorption and transient and undesirable high blood drug concentrations observed after administration. Furthermore, with oral administration, many side effects have been reported, such as gastrointestinal disorders, vomiting sensation, and loss of appetite. Furthermore, among patients with schizophrenia, it is believed that approximately 75% actually have difficulty taking oral preparations on a regular basis. Therefore, in order to solve these problems regarding oral administration and prepare a preparation that patients can take easily, safely and sustainably, a method of applying transdermal patch preparation is provided, which can solve the above-mentioned problems regarding oral administration, and has the advantages of reducing administration frequency, improving compliance, and making it easier for drug application or withdrawal. Therefore, transdermal administration patches are expected to be a useful route of administration.

In practice, to produce antipsychotic effects or to alleviate behavioral disturbances associated with neurodegenerative diseases, risperidone is usually administered as tablets, oral solution, or intramuscular injection solution. For a number of reasons, it would be desirable to administer risperidone in a non-invasive manner, specifically as a transdermal patch that would allow controlled release of the drug.

Risperidone is a highly effective drug with a relatively narrow therapeutic index. It may lead to adverse side effects in response to overdose, most notably extrapyramidal syndrome (EPS) and, to a lesser extent, hypotension (due to peripheral alpha-adrenergic activity). To produce antipsychotic effects in patients, the total daily dose of risperidone ranges from about 2 to about 8 mg. To alleviate behavioral disturbances associated with neurodegenerative diseases, the total daily dose is generally small, usually in the range of about 0.5 to about 2 mg. For interindividual variability and for additional drug therapy, dose titration in patients is required to obtain an effective dose.

Risperidone is metabolized to 9-hydroxyrisperidone, which has pharmacological properties and potency comparable to the parent drug risperidone, but has a longer elimination half-life. Risperidone is more rapidly distributed into brain tissue for elimination than its metabolite 9-hydroxyrisperidone.

Isoquinoline-type genetic polymorphisms play a unique role in the metabolism of risperidone. Based on metabolic rate, humans can be classified as poor, intermediate, or extensive metabolizers. The metabolic rate is defined as the ratio of the urinary recovery of isoquinoline to the urinary recovery of metabolite 4-hydroxyisoquinoline of isoquinoline within 8 hours after oral ingestion of 10 mg of isoquinoline. In the Easterners, more than 99% of the population can be phenotyped because extensive metabolizers and poor metabolizers are quite rare. However, within the Caucasian race, only approximately 90% of the population can be phenotyped as extensive or intermediate metabolizers. About 10% of the population has poor metabolism and insufficient levels of isoquinoline hydroxylase. The duration of action and peak plasma levels of the active agents (risperidone and 9-hydroxyrisperidone) are largely dependent on the rate of isoquinoline metabolism in human subjects receiving risperidone. More specifically, high transient peak levels of risperidone may be achieved in poor metabolizers when the total daily dose is administered at a single dose. This may cause undesirable side effects such as extrapyramidal syndrome (EPS) and hypotension (due to adrenergic effects). In general, the rapid distribution of risperidone in plasma and brain tissue indicates that the drug can be better administered periodically in divided doses, and it is best to administer risperidone continuously at a controlled rate to avoid the possibility of excessive peak levels (and side effects) while maintaining a clinically effective drug level. If risperidone could be administered transdermally, then a rate-controlled transdermal administration system would clearly provide a practical solution to the above problems. Furthermore, it is desirable that the transdermal administration system can deliver a drug at a substantially constant rate for at least about 24 hours while keeping the amount of unused and depleted drug in the system to a minimum. Given the pharmacological characteristics of risperidone, continuous delivery of therapeutic amounts of risperidone by a transdermal administration system that relies primarily on skin permeability to control the rate of drug penetration is not possible. There is a need to develop a transdermal administration system that controls the rate of drug penetration, which itself controls the maximum rate of drug delivery through the skin.

CN 101366705 B and U.S. Pat. No. 8,431,152 B1 describe the preparation of corresponding patch preparations by dissolving a certain dose of risperidone in an organic solvent, and are not expected to contain risperidone dispersed in the matrix in a crystalline form. CN 101366705 B does not disclose that the patch can be applied continuously at a therapeutically effective dose and at a constant rate for 3 days (72 hours) or more, nor does it disclose that the patch contains risperidone dispersed in the matrix in a crystalline form. Example 4 and FIG. 6 of the aforementioned patent show that there is no linear relationship between the cumulative release rate and the increasing dosage of risperidone. According to the paper “Study on the Risperidone Transdermal Administration System” published by Chen Xiaojin, the inventor of the aforementioned patent, oleic acid is not the most preferred penetration enhancer for risperidone patch (see the last line on page 29), and azone, dodecanol and propylene glycol are the preferred penetration enhancers. However, azone has suspicious pharmacological activity or its safety is questionable. Dodecanol is a skin irritant. Propylene glycol is the solvent used to dissolve risperidone.

Therefore, there is an urgent need for a transdermal administration system of risperidone or a pharmaceutically acceptable salt thereof, which can deliver risperidone or a pharmaceutically acceptable salt thereof continuously at a therapeutically effective amount of blood drug concentration within 24 hours to 14 days, thereby improving the medication compliance of patients and providing long-term effective treatment for schizophrenia patients.

### SUMMARY OF THE INVENTION

The present invention has found that it is difficult to achieve sustained drug release to a patient in a rate-controlled manner when administering a transdermal administration system containing risperidone, let alone to continuously deliver risperidone into the patient at a substantially constant rate within 24 hours to 14 days to maintain a therapeutically effective amount of blood drug concentration in the patient.

The present invention has now unexpectedly discovered that both lauryl lactate and long chain fatty alcohols are effective skin penetration enhancers for risperidone. They have no pharmacological activity and are non-irritating to the skin. The present invention has also unexpectedly found that microcrystalline risperidone without dissolution using solvents can provide long-term sustained transdermal delivery at a constant rate. When the transdermal administration system contains risperidone crystal form A and skin penetration enhancer (preferably lauryl lactate, long-chain fatty alcohol such as oleyl alcohol), it has good stability, and can continuously deliver drugs in a controlled manner at a constant rate within a continuous period of 3 days, 7 days, 10 days and 14 days, and the transdermal amount of risperidone is maintained above 65% of the maximum transdermal amount within day 3-day 7 after administration. The system continuously supplies risperidone to the skin, and the skin penetration enhancer allows the drug to penetrate the skin into the circulatory system at therapeutically effective doses.

The present invention also found that, the skin flux can be increased by controlling the dosage of the skin penetration enhancer and risperidone crystal form A, which is beneficial to achieving the above technical effects.

The present invention also found that, the skin flux can be increased by including undissolved risperidone crystal form A in the matrix layer of the risperidone administration system of the present invention, which is beneficial to achieving the above technical effects. The present invention also found that the skin adhesion of the transdermal administration system can be improved by adjusting the amounts of the pressure-sensitive adhesive and the cohesion promoter relative to the total weight of the matrix layer, so that the system can be well tolerated by the skin.

Penetration enhancers accelerate drug release from transdermal administration systems, which has the opposite effect to the purpose of sustained release. The present invention also found that a specific matrix combination can obtain a transdermal administration system of risperidone in both crystalline and non-crystalline, which is beneficial to the sustained delivery of risperidone at a constant rate.

Therefore, the object of the present invention is to provide a matrix-type risperidone transdermal administration system that can continuously deliver risperidone or a pharmaceutically acceptable salt thereof at a constant rate within 24 hours to 14 days at a therapeutically effective amount of blood drug concentration.

Another object of the present invention is to provide a depot-type risperidone transdermal administration system that can continuously deliver risperidone or a pharmaceutically acceptable salt thereof within 24 hours to 14 days at a therapeutically effective amount of blood drug concentration.

Another object of the present invention is to provide a method for preparing a matrix-type risperidone transdermal administration system, which can continuously deliver risperidone or a pharmaceutically acceptable salt thereof within 24 hours to 14 days at a therapeutically effective amount of blood drug concentration.

Another object of the present invention is to provide the use of a therapeutically effective amount of a risperidone transdermal administration system in preparing a drug for treating or preventing schizophrenia, mania and dementia, which includes administering a therapeutically effective amount of a stable risperidone transdermal administration system to a subject in need.

Another object of the present invention is to provide the use of a therapeutically effective amount of a risperidone transdermal administration system in preparing a drug for treating or preventing positive and negative symptoms of schizophrenia, which includes administering a therapeutically effective amount of a stable risperidone transdermal administration system to a subject in need.

Another object of the present invention is to provide the use of a therapeutically effective amount of a stable risperidone transdermal administration system in preparing a drug for treating or preventing hallucinations, delusions, emotional withdrawal and emotional retardation symptoms of schizophrenia, which includes administering a therapeutically effective amount of a stable risperidone transdermal administration system to a subject in need.

Another object of the present invention is to provide a method of treating or preventing schizophrenia, mania and dementia, which includes administering a therapeutically effective amount of a stable risperidone transdermal administration system to a subject in need.

Another object of the present invention is to provide a method of treating or preventing positive and negative symptoms of schizophrenia, which includes administering a therapeutically effective amount of a stable risperidone transdermal administration system to a subject in need.

Another object of the present invention is to provide a method of treating or preventing hallucinations, delusions, emotional withdrawal and emotional retardation symptoms of schizophrenia, which includes administering a therapeutically effective amount of a stable risperidone transdermal administration system to a subject in need.

### BRIEF DESCRIPTION OF THE DRAWINGS

FIG. 1 shows a schematic diagram of the backing layer, matrix layer and release liner layer in the three-layer matrix-type transdermal administration system of the present invention.

FIG. 2 shows a schematic diagram of the backing layer, matrix layer, skin-contacting adhesive layer and release liner layer in the four-layer matrix-type transdermal administration system of the present invention.

FIG. 3 shows a schematic diagram of the backing layer, matrix layer, semipermeable membrane or machine-woven fabric layer, skin-contacting adhesive layer and release liner layer in the five-layer matrix-type transdermal administration system of the present invention.

FIGS. 4A-4F show schematic diagrams of matrix-type transdermal administration systems using overlapping adhesive films.

FIG. 5 shows the skin flux test results of comparative examples 1 to 6.

FIG. 6 shows the skin flux test results of examples 1 to 3.

FIG. 7 shows the skin flux test results of example 4.

FIG. 8 shows the XRPD spectrum of risperidone crystal form A.

FIG. 9 shows the XRPD spectrum of the transdermal patch preparation of crystalline risperidone.

FIG. 10 shows the skin flux test results of example 5 and example 8.

FIG. 11 shows the skin flux test results of example 9 and example 12.

FIG. 12 shows the skin flux test results of examples 26 to 33 and comparative example 7.

FIG. 13 show schematic diagrams of matrix-type transdermal administration systems using overlapping adhesive films in which the separation layer and the drug matrix layer/skin adhesive layer are cut into small pieces.

FIG. 14 shows a schematic view in which the separation layer and the drug matrix layer are not cut into small pieces.

FIG. 15 shows a schematic diagram in which the separation layer and the drug matrix layer are divided into 4 small pieces of 12.5 cm²; There is one connection point between each small piece, and the intersecting black lines are the cut separation layer and drug matrix layer.

FIG. 16 shows a schematic diagram in which the separation layer and the drug matrix layer are divided into 4 small pieces of 12.5 cm²; There is two connection points between each small piece, and the intersecting black lines are the cut separation layer and drug matrix layer.

FIG. 17 shows a schematic diagram in which the separation layer and the drug matrix layer are divided into 4 small pieces of 12.5 cm²; There is no connection point between each small piece, and the intersecting black lines are the cut separation layer and drug matrix layer.

FIG. 18 shows the pieces without connection points of FIG. 17. When the inner patch (separation layer and matrix layer) moves forward, the 4 parts of the inner patch are separated from each other and arranged in disorder.

FIG. 19 shows the small pieces with two connection points shown in FIG. 16. When the inner patch (separation layer and matrix layer) moves forward, the 4 small pieces of the inner patch are connected together in order.

FIG. 20 shows the skin flux test results of examples 42 to 45.

### DETAILED DESCRIPTION OF EMBODIMENTS

In one aspect, the present invention provides a risperidone transdermal administration system, which includes:
1) a backing layer;
2) a matrix layer, which contains risperidone dispersed in the matrix layer in crystalline and non-crystalline, a skin penetration enhancer and a pressure-sensitive adhesive; and
3) a release liner layer.

In some embodiments, the crystalline form of risperidone in the risperidone transdermal administration system is crystalline form A, preferably micronized risperidone crystalline form A.

In some embodiments, the skin penetration enhancer is selected from one or more of C₁-C₃₀ fatty acids, fatty esters, or fatty alcohols;
preferably, the fatty acids are selected from C₁₂-C₂₂ unsaturated fatty acids, more preferably oleic acid;
preferably, the fatty esters are selected from esters produced by C₁-C₃₀ fatty acids and C₁-C₃₀ alcohols in combination, more preferably lauryl lactate;
preferably, the fatty alcohols are selected from C₁₃-C₃₀ alkenyl alcohols, more preferably oleyl alcohol.

In some embodiments, the matrix layer in the risperidone transdermal administration system includes the following components relative to the total weight of the matrix layer:
1) risperidone at a total amount of 9.5-15%, preferably 9.5-15% or 10-12%;
2) the skin penetration enhancer at an amount of 1-50%, preferably 2-30%, 8.5-30%, 8.5-25%, 8.5-15%; more preferably, the C₁-C₃₀ fatty acids at an amount of 8.5-15%, the fatty esters at an amount of 1-10%, and the fatty alcohols at an amount of 1-15%; and most preferably, oleic acid at an amount of 8.5-15%, lauryl lactate at an amount of 1-10%, 1-6% or 3-5%, propylene glycol monolaurate at an amount of 2-15%, 3-15%, 5-15%, 2-10%, 3-10%, 5-10%, C₁₃-C₃₀ alkenyl alcohol at an amount of 1-15%, 2-15%, 5-15% or 8-15%;
3) the pressure-sensitive adhesive at an amount of 50-82%, preferably 50-80% or 55-80%;
the total amount of all component in the matrix layer is 100%.

In some embodiments, the matrix layer in the risperidone transdermal administration system consists of the following components relative to the total weight of the matrix layer:
1) risperidone at a total amount of 9.5-15%, preferably 9.5-15% or 10-12%;
2) the skin penetration enhancer at an amount of 1-50%, preferably 2-30%, 8.5-30%, 8.5-25%, 8.5-15%; more preferably, the C₁-C₃₀ fatty acids at an amount of 8.5-15%, the fatty esters at an amount of 1-10%, and the fatty alcohols at an amount of 1-15%; and most preferably, oleic acid at an amount of 8.5-15%, lauryl lactate at an amount of 1-10%, 1-6% or 3-5%, C₁₃-C₃₀ alkenyl alcohol at an amount of 1-15%, 5-15% or 8-15%;
3) the pressure-sensitive adhesive at an amount of 50-82%, preferably 50-80% or 55-80%;
4) an antioxidant at an amount of 0%-1%, preferably 0.05%-0.5% or 0.1%-0.3%;
5) a cohesion promoter at an amount of 0-30%, preferably 1-5%;
6) a tackifier at an amount of 0-40%;
7) a plasticizer at an amount of 0-40%;
8) an additional penetration enhancer at an amount of 0-10%;
the total amount of all component in the matrix layer is 100%.

The additional penetration enhancer refers to another penetration enhancer different from 2).

In some embodiments, the pressure-sensitive adhesive is selected from one or more of acrylic adhesives, organosilicon adhesives, acrylic-organosilicon copolymer adhesives, polybutylene adhesives, styrene-isoprene-butylene copolymers, and styrene-butadiene-styrene copolymers.

In some embodiments, the cohesion promoter is selected from crospovidone, Eudragit E, Eudragit EPO, Eudragit S, Eudragit R. Plastoid B, and mixtures thereof. In some embodiments, the cohesion promoter is povidone K90, Eudragit EPO, and combinations of povidone K90 and Eudragit EPO, crospovidone CL-M and Eudragit EPO, povidone K90 and crospovidone CL-M and Eudragit EPO, povidone K90 and Plastoid B, crospovidone CL-M and Plastoid B, povidone K90 and crospovidone CL-M, the preferred combination is povidone K90 and Eudragit EPO.

In some embodiments, the antioxidant is selected from tocopherol, tocopheryl acetate, potassium metabisulfite, sodium metabisulfite, sodium bisulfite, sodium sulfite, propyl gallate, thioglycerol, sodium thiosulfate, sodium dioxide, sodium formaldehyde sulfoxylate, butylated hydroxytoluene (BHT); preferably, alpha-tocopherol (i.e. vitamin E), or a combination of alpha-tocopherol and butylated hydroxytoluene (BHT). In some embodiments, the further chelating agent as a synergistic antioxidant is selected from citric acid, tartaric acid, calcium disodium edetate, disodium edetate, and EDTA.

In some embodiments, the tackifier is selected from polybutenes, terpenes, and mixtures thereof.

In some embodiments, the plasticizer is selected from mineral oil, silicone oil, triethyl citrate, and mixtures thereof.

In some embodiments, the amount of the matrix layer in the risperidone transdermal administration system is from 30 g/m² to 700 g/m². In some embodiments, the amount of the matrix layer is 30 GSM to 700 GSM, 50 GSM to 700 GSM, 30 GSM to 100 GSM, 40 GSM to 150 GSM, 75 GSM to 150 GSM, 150 GSM to 300 GSM, or 350 GSM to 700 GSM. The specific amount of the matrix layer is selected from 30 GSM, 50 GSM, 55 GSM, 75 GSM, 100 GSM, 125 GSM, 150 GSM, 175 GSM or 200 GSM.

In some embodiments, during the preparation process of the risperidone transdermal administration system, risperidone is added in the form of micronized risperidone crystal form A, preferably the particle size of the micronized risperidone is less than or equal to 20 μm. In some embodiments, 90% or more of the particles have an average diameter of 20 μm or less, more preferably from 0.5 nm to 20 μm, and particularly preferably from 0.5 nm to 15 μm.

In some embodiments, aolubilizers or solvents are also used in the preparation of the matrix layer of risperidone transdermal administration system. Solubilizers or solvents have the effect of reducing viscosity (i.e. viscosity reducers). The solubilizer or solvent is selected from C₁-C₆ alkyl alcohol, n-heptane, ethyl acetate, toluene and mixtures thereof, preferably ethanol, isopropanol, n-heptane or ethyl acetate.

In some embodiments, a skin contact adhesive layer is added over the matrix layer (FIG. 2).

The risperidone transdermal administration system includes:
1) a backing layer;
2) a matrix layer, which contains risperidone dispersed in the matrix layer in crystalline and non-crystalline, a skin penetration enhancer and a pressure-sensitive adhesive;
3) a skin-contacting adhesive layer; and
4) a release liner layer.

In some embodiments, a rate control film layer (film or machine-woven fabric in FIG. 3) is added between the drug matrix reservoir layer and the skin contact adhesive layer. The risperidone transdermal administration system includes:
1) a backing layer;
2) a matrix layer, which contains risperidone dispersed in the matrix layer in crystalline and non-crystalline, a skin penetration enhancer and a pressure-sensitive adhesive;
3) a semipermeable membrane or machine-woven fabric layer;
4) a skin-contacting adhesive layer; and
5) a release liner layer.

In some embodiments, the skin-contacting adhesive layer includes a pressure-sensitive adhesive and optionally risperidone and optionally other excipients selected from one or more of skin penetration enhancers, cohesion promoters, antioxidants, anti-skin irritation agents, tackifiers, plasticizers, solubilizers, solvents and desiccants.

In some embodiments, the risperidone transdermal administration system includes:
1) a backing layer;
2) an overlapping adhesive film layer, preferably the material of the overlapping adhesive film layer is selected from one or more of siloxane, polyisobutylene, or styrene-isoprene-styrene copolymer; preferably, the amount of the overlapping adhesive film layer is 50-110 GSM;
3) a separation layer;
4) a matrix layer, which contains risperidone dispersed in the matrix layer in crystalline and non-crystalline, a skin penetration enhancer and a pressure-sensitive adhesive; and
5) a release liner layer.

In some embodiments, the overlapping adhesive film layer of the risperidone transdermal administration system includes an adhesive, wherein the adhesive of the overlapping adhesive film layer is the same as or different from the adhesive of the drug matrix layer. Preferably, the solubility of a drug in the adhesive of the overlapping adhesive film layer is the same as or less than that of the drug in the adhesive of the matrix layer. More preferably, the solubility of the drug in the adhesive of the overlapping adhesive film layer is less than that of the drug in the adhesive of the matrix layer.

In some embodiments, the overlapping adhesive film layer can be one layer, two layers or multiple layers. Preferably, a first overlapping adhesive film layer close to the backing layer can prevent other overlapping adhesive film layers away from the backing layer from migrating to the backing layer or the side of the backing layer away from the skin.

In some embodiments, the adhesive of the first overlapping adhesive film layer is selected from Duro-Tak 387-2516, Duro-Tak 387-2287, Duro-Tak 387-4287, Duro-Tak 87-2051, Duro-Tak 87-2052, Duro-Tak 87-2054, Duro-Tak 87-2852 or mixtures thereof. In some embodiments, the adhesive of the second overlapping adhesive film layer is selected from polyisobutylene, styrene-isoprene-styrene copolymer, dimethylsiloxane, or mixtures thereof.

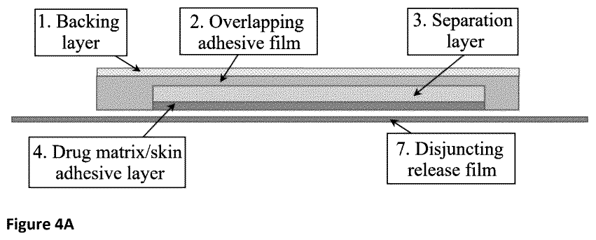

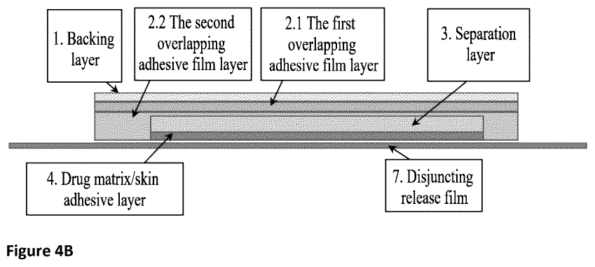

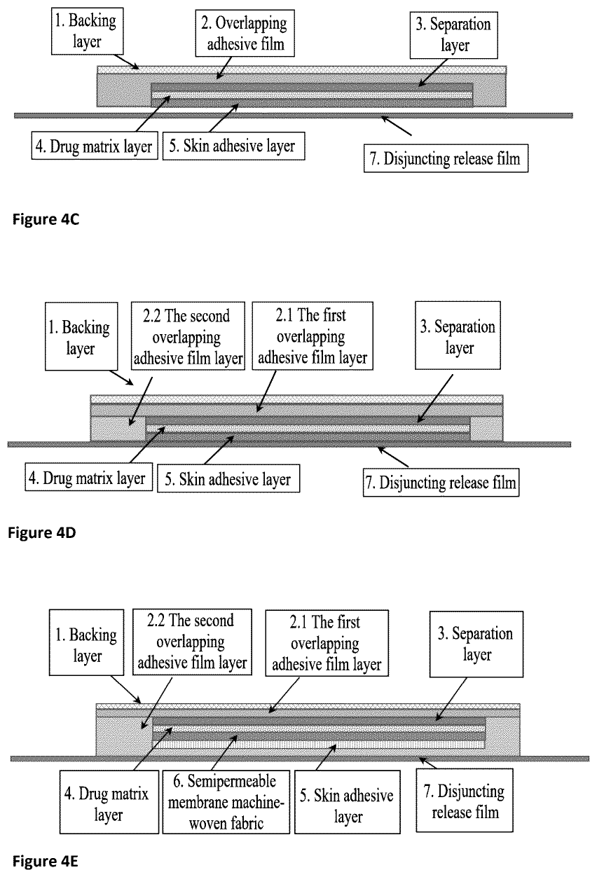

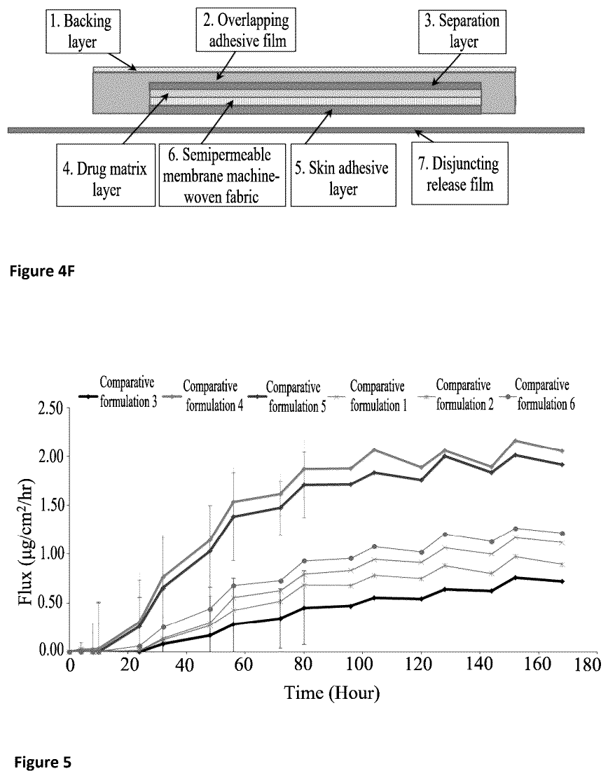

In some embodiments, the risperidone transdermal administration system comprising overlapping adhesive film layers, sequentially from a backing layer to a disjuncting layer, includes: a backing layer, one or more overlapping adhesive film layers, a separation layer, a matrix layer, and a release liner layer (as shown in FIG. 4A); the backing layer, the one or more overlapping adhesive film layers and the release liner layer extend all around beyond the separation layer and the matrix layer; During application, the disjuncting layer is removed and the matrix layer in contact with the skin is sealed to the skin by one or more overlapping adhesive film layers.

In some embodiments, in the risperidone transdermal administration system comprising overlapping adhesive film layers, the overlapping adhesive film is one layer (as shown in FIGS. 4A, 4C and 4F), and the risperidone transdermal administration system, sequentially from a backing layer to a disjuncting layer, includes:
i. a backing layer, an overlapping adhesive film layer, a separation layer, a matrix layer, and a release liner layer, and the backing layer, the overlapping adhesive film layer, and the release liner layer extend all around beyond the separation layer and the matrix layer; the structure illustrated in FIG. 4A is preferred; or
ii. a backing layer, an overlapping adhesive film layer, a separation layer, a matrix layer, a skin adhesive layer, and a release liner layer, and the backing layer, the overlapping adhesive film layer and the release liner layer extend all around beyond the separation layer, the matrix layer and the skin adhesive layer; the structure illustrated in FIG. 4C is preferred; or
iii. a backing layer, an overlapping adhesive film layer, a separation layer, a matrix layer, a semipermeable membrane layer, a skin adhesive layer, and a release liner layer; and the backing layer, the overlapping adhesive film layer and the release liner layer extend all around beyond the separation layer, the matrix layer, the semipermeable membrane layer and skin adhesive layer; the structure illustrated in FIG. 4F is preferred.

In some embodiments, in the risperidone transdermal administration system comprising overlapping adhesive film layers, the overlapping adhesive film is two layers (as shown in FIGS. 4B, 4D, 4E and 13); During application, the disjuncting layer is removed and the matrix layer in contact with the skin is sealed to the skin by the second overlapping adhesive film layer; the risperidone transdermal administration system, from a backing layer to a disjuncting layer, includes:
i. a backing layer, a first overlapping adhesive film layer, a second overlapping adhesive film layer, a separation layer, a matrix layer, and a release liner layer, and the backing layer, the first overlapping adhesive film layer, the second overlapping adhesive film layer and the release liner layer extend all around beyond the separation layer and the matrix layer; the structure illustrated in FIG. 4B is preferred; or
ii. a backing layer, a first overlapping adhesive film layer, a separation layer, a matrix layer, a skin adhesive layer, a release liner layer, and a backing layer, and the first overlapping adhesive film layer and the release liner layer extend all around beyond the separation layer, the matrix layer and the skin adhesive layer, the second overlapping adhesive film layer is filled between the first overlapping adhesive film layer and the release liner layer, the structure illustrated in FIG. 4D is preferred; or
iii. a backing layer, a first overlapping adhesive film layer, a separation layer, a matrix layer, a semipermeable membrane layer, a skin adhesive layer, a second overlapping adhesive film layer, and a release liner layer, and the backing layer, the first overlapping adhesive film layer, the second overlapping adhesive film layer and the release liner layer extend all around beyond the separation layer, the matrix layer, the semipermeable membrane layer and the skin adhesive layer, and the second overlapping adhesive film layer extends toward the first overlapping adhesive film layer and wraps the separation layer, the matrix layer, the semipermeable membrane layer and the skin adhesive layer; the structure illustrated in FIG. 4E is preferred.

In some embodiments, the amount of the first overlapping adhesive film layer is 25-60 GSM; the amount of the second overlapping adhesive film layer is 25-60 GSM, preferably, the amount of the first overlapping adhesive film layer is 50-60 GSM; the amount of the second overlapping adhesive film layer is 50-60 GSM. In some specific embodiments, the amount of the first overlapping adhesive film layer is 25, 50, 55 or 60 GSM; The amount of the second overlapping adhesive film layer is 25, 50, 55 or 60 GSM.

In some embodiments, the backing layer is composed of a polymer elastic film, a polymer fabric, a bidirectional or multi-directional elastic nonwoven fabric, a stretchable polymer film, a stretchable machine-woven fabric, or a stretchable nonwoven fabric. In some embodiments, the backing layer is selected from polyester, polyethylene, polypropylene, polyvinyl chloride, polyethylene vinyl acetate, or polyurethane, preferably KOB051 and KOB053.

In some embodiments, the separation layer is selected from a flexible occlusive single-layer or multi-layer polymeric film. The polymer of the separation layer is selected from polyolefins, polyesters, polyethylene, polyvinylidene chloride or polyurethane. Preferably, the separation layer further includes an aluminum film.

In some embodiments, the separation layer includes a plurality of small pieces that are partially connected to each other, and the matrix layer includes a plurality of small pieces that are fully or partially connected. Preferably, the plurality of separation layer small pieces connected to each other in the separation layer are symmetrical or asymmetrical, the same or different. The plurality of small pieces connected to each other in the matrix layer are symmetrical or asymmetrical, the same or different. Further preferably, a part of the connection points of the separation layer and a part of the connection points of the matrix layer are all aligned or partially aligned.

In some embodiments, there are one or more connection points between adjacent pieces in the separation layer or matrix layer; 1, 2, 3, 4 or 5 connection points are preferred, and the length of each connection point is independently selected from the range of 0.5-3 mm.

In some embodiments, the overlapping separation layer pieces and the matrix layer pieces have the same shape, and the number of the pieces in each transdermal administration system unit is selected from 1 to 10.

In some embodiments, each small piece has an area of 1-25 cm², preferably 1 cm², 4 cm², 6 cm², 8 cm², 9 cm², 10 cm², 12.5 cm², 16 cm², or 25 cm².

In some embodiments, the matrix layer has a weight of 30 GSM to 700 GSM, 50 GSM to 700 GSM, 30 GSM to 100 GSM, 40 GSM to 150 GSM, 150 GSM to 300 GSM, or 350 GSM to 700 GSM.

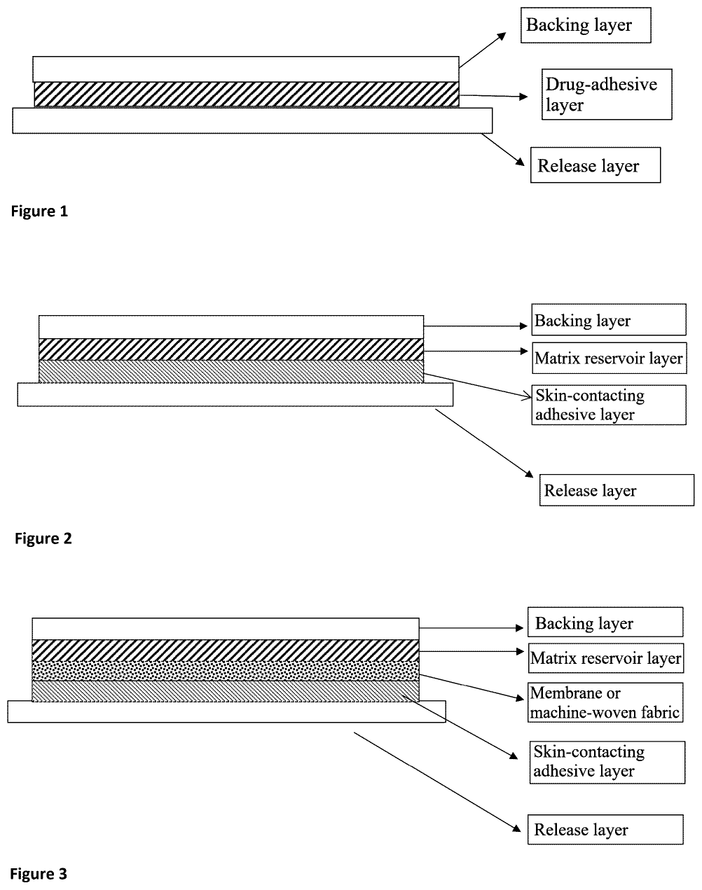

On the other hand, the present invention provides a method of preparing a stable matrix-type risperidone transdermal administration system (as shown in FIG. 1), which includes the following steps:
step 1. mixing a skin penetration enhancer and optionally a cohesion promoter, an antioxidant, an anti-skin irritation agent, a tackifier, a plasticizer, a solubilizer, a solvent and a desiccant to obtain blend 1;
step 2. mixing the blend 1 and a pressure-sensitive adhesive to obtain blend 2;
step 3. adding micronized risperidone crystal form A or a pharmaceutically acceptable salt thereof to the blend 2 from step 2, and stirring the resulting mixture until risperidone or the pharmaceutically acceptable salt thereof is evenly suspended to obtain a wet drug mixture;
step 4. coating the wet drug mixture on a release liner layer;
step 5. drying to remove the solvent and the solubilizer to obtain a release liner layer/drug matrix layer laminated material; and
step 6. laminating the release liner layer/drug matrix layer laminated material to the backing layer to form a release liner layer/drug matrix layer/backing layer composite film.

In some embodiments, the present invention provides a method of preparing a matrix-type risperidone transdermal administration system (as shown in FIG. 2), comprising the following steps:
step 1. preparing a release liner layer/matrix layer/backing layer composite film according to the aforementioned steps 1 to 6;
step 2. preparing a solution or a suspension of a skin-contacting adhesive layer containing a pressure-sensitive adhesive and optionally risperidone crystal form A or a pharmaceutically acceptable salt thereof and optionally other excipients, coating same to a release liner layer and drying to form a release liner layer/skin-contacting adhesive layer laminated material; and step 3. removing the release liner layer from the release liner layer/matrix layer/backing layer composite film, and laminating the adhesive layer side of the release liner layer/skin-contacting adhesive layer of step 1 to the matrix layer to form a release liner layer/skin-contacting adhesive layer/matrix layer/backing layer composite film.

In some embodiments, the present invention provides a method of preparing a matrix-type risperidone transdermal administration system (as shown in FIG. 3), comprising the following steps:
step 1. preparing a release liner layer/matrix layer/backing layer composite film according to the aforementioned steps 1 to 6 of the claims;
step 2. preparing a solution or a suspension of a skin-contacting adhesive layer containing a pressure-sensitive adhesive and optionally risperidone crystal form A or a pharmaceutically acceptable salt thereof and optionally other excipients, coating same to a release liner layer and drying to form a release liner layer/skin-contacting adhesive layer laminated material, and laminating the adhesive layer side to a semipermeable membrane or machine-woven fabric layer; and
step 3. removing the release liner layer from the release liner layer/matrix layer/backing layer composite film, and laminating the semipermeable membrane or machine-woven fabric layer side to the matrix layer to form a release liner layer/skin-contacting adhesive layer/semipermeable membrane or machine-woven fabric layer/matrix layer/backing layer composite film.

In some embodiments, the present invention provides a risperidone transdermal administration system (as shown in FIG. 4A), comprising the following steps:
step A: coating the adhesive of the overlapping adhesive film layer on the release film, and after drying, compounding same to the backing layer to obtain a backing layer/overlapping adhesive film/release film composite;
step B: preparing the release liner layer/drug matrix layer laminated material according to steps 1 to 5 of claim 32, and laminating the release liner layer/drug matrix layer laminated material to the separation layer to form a release liner layer/drug matrix layer/separation layer film;
step C: removing the release film of the backing layer/overlapping adhesive film/release film composite of step A, and applying the separation layer of the release liner layer/drug matrix layer/separation layer film of step B to the overlapping adhesive film;
step D: removing the release film from the drug matrix layer and applying an oversized release film on the drug matrix layer and the overlapping adhesive film layer;
step E: die-cutting the final patch so that the overlapping adhesive film layer extends beyond the separation layer and the drug adhesive layer in each direction.

In some embodiments, the present invention provides a risperidone transdermal administration system (as shown in FIG. 4C), comprising the following steps:
step A: coating the adhesive of the overlapping adhesive film layer on the release film, and after drying, compounding same to the backing layer to obtain a backing layer/overlapping adhesive film/release film composite;
step B: preparing a release liner layer/skin-contacting adhesive layer/drug matrix layer drug/separation layer composite film;
step C: removing the release film of the separation layer/overlapping adhesive film/release film composite of step A, and applying the separation layer of the release liner layer/skin-contacting adhesive layer/matrix layer/separation layer composite film formed in step B to the overlapping adhesive film;
step D: removing the release film from the skin-contacting adhesive layer and applying an oversized release film to the skin-contacting adhesive layer and the overlapping adhesive film layer;
step E: die-cutting the final patch so that the overlapping adhesive film layer extends beyond the separation layer, the matrix layer and the skin adhesive layer in each direction; preferably, the structure of FIG. 4C is formed.

In some embodiments, the present invention provides a risperidone transdermal administration system (as shown in FIG. 4F), comprising the following steps:
step A: coating the adhesive of the overlapping adhesive film layer on the release film, and after drying, compounding same to the backing layer to obtain a backing layer/overlapping adhesive film/release film composite;
step B: preparing the release liner layer/skin-contacting adhesive layer/semipermeable membrane or machine-woven fabric layer/drug matrix layer/separation layer composite film;
Step C: removing the release film of the backing layer/overlapping adhesive film/release film composite of step A, and applying the separation layer of the release liner layer/skin-contacting adhesive layer/semipermeable membrane or machine-woven fabric layer/drug matrix layer/separation layer composite film of step B to the overlapping adhesive film;
Step D: removing the release film from the skin-contacting adhesive layer and applying an oversized release film to the skin-contacting adhesive layer and the overlapping adhesive film layer;
step E: die-cutting the final patch so that the backing layer, the overlapping adhesive film layer and the release liner layer extend all around beyond the separation layer, the matrix layer, the semipermeable membrane or machine-woven fabric layer and the skin adhesive layer; preferably, the structure of FIG. 4F is formed.

In some embodiments, the present invention provides a risperidone transdermal administration system (as shown in FIGS. 4B, 4D, 4E), comprising the following steps:
step A: coating the adhesive of the first overlapping adhesive film layer on the release film, and after drying, compounding same to the backing layer to obtain a backing layer/first overlapping adhesive film/release film composite film; coating the adhesive of the second overlapping adhesive film layer on the release film, and after drying, compounding same to the first overlapping adhesive film layer from which the release film is removed to obtain a backing layer/first overlapping adhesive film/second overlapping adhesive film/release film composite film.
step B: preparing a release liner layer/drug matrix layer/separation layer film (FIG. 4B), a release liner layer/skin adhesive layer/drug matrix layer/separation layer film (FIG. 4D), or a release liner layer/skin adhesive layer/semipermeable membrane machine-woven fabric layer/drug matrix layer/separation layer film (FIG. 4E);
step C: removing the release film of the backing layer/first overlapping adhesive film/second overlapping adhesive film/release film composite film of step A, and applying the separation layer of step B to the second overlapping adhesive film layer;
step D: removing the remaining release film of step C and applying an oversized release film on the side away from the backing layer;
step E: die-cutting the final patch so that the second overlapping adhesive film layer extends beyond the separation layer, the drug matrix layer, and optionally the skin adhesive layer and the semipermeable membrane machine-woven fabric layer in each direction; preferably, the structures of FIGS. 4B, 4D, and 4E are obtained.

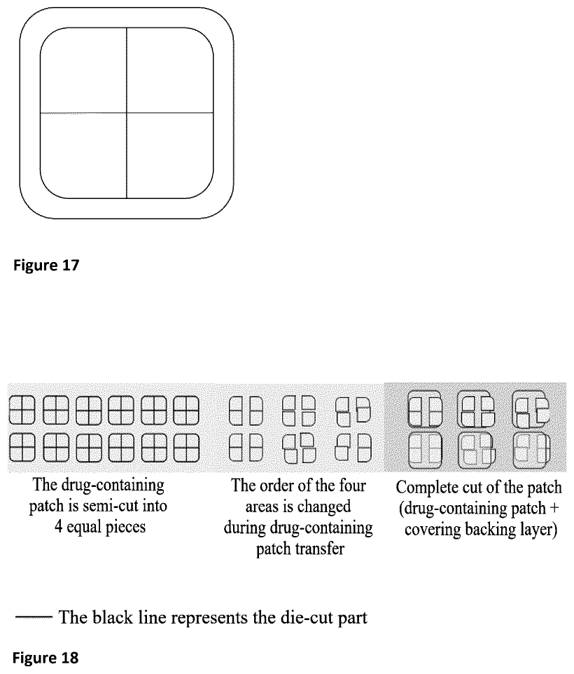

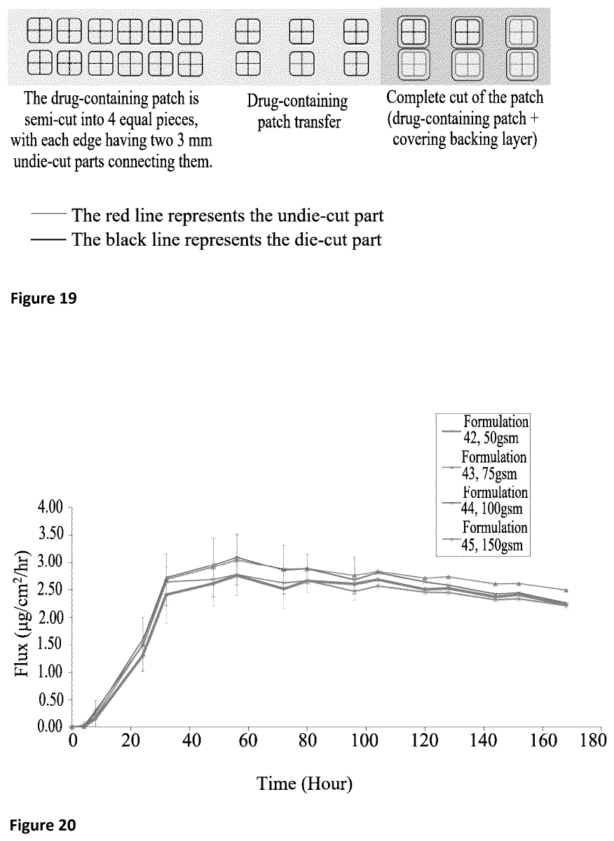

In some embodiments, after cutting the composite film prepared in the aforementioned B into a plurality of small pieces which are partially connected, the structure of the small pieces is obtained (as shown in FIGS. 13 and 15-19); The structure in step C improves the arrangement order of the composite film in step B during transfer (as shown in FIGS. 18-19).

In some embodiments, the wet drug mixture used to prepare the matrix layer contains undissolved risperidone crystal form A.

In some embodiments, the solvent in step 1 or step B is selected from C₁-C₆ alkyl alcohols, n-heptane, ethyl acetate, toluene, and mixtures thereof; preferably, ethanol, isopropanol, n-heptane or ethyl acetate.

In some embodiments, the risperidone transdermal administration system is packaged in a heat-sealed bag.

In another aspect, the present invention provides a use of a therapeutically effective amount of risperidone transdermal administration system in the preparation of a drug for treating or preventing schizophrenia, mania and dementia.

In another aspect, the present invention provides a use of a risperidone transdermal administration system in the preparation of a drug for treating or preventing positive and negative symptoms of schizophrenia. In some embodiments, the positive and negative symptoms of schizophrenia include hallucinations, delusions, and emotional withdrawal and blunted affect.

In another aspect, the present invention provides a method of treating or preventing schizophrenia, mania and dementia, which includes administering a therapeutically effective amount of a risperidone transdermal administration system to a subject in need.

In another aspect, the present invention provides a method of treating or preventing positive and negative symptoms of schizophrenia, which includes administering a therapeutically effective amount of a risperidone transdermal administration system to a subject in need. In some embodiments, the positive and negative symptoms of schizophrenia include hallucinations, delusions, and emotional withdrawal and blunted affect.

In some embodiments, the risperidone transdermal administration system is administered every 1 day, every 3 days, every 7 days, every 10 days, or every 14 days.

In some embodiments, the risperidone transdermal administration system continuously delivers risperidone or a pharmaceutically acceptable salt thereof at a therapeutically effective amount of blood drug concentration within 24 hours to 14 days.

In some embodiments, the risperidone transdermal administration system continuously delivers risperidone into the patient at a substantially constant rate within 24 hours to 14 days, preferably within 24 hours to 7 days.

In some embodiments, the maximum transdermal amount of risperidone occurs within 24-36 hours after administration of the risperidone transdermal administration system, and the transdermal amount of risperidone is maintained more than 65% of the maximum transdermal amount during day 3-day 7 after the administration; preferably, maintained 65%-90% of the maximum transdermal amount of risperidone; more preferably, maintained 75%-85% of the maximum transdermal amount of risperidone.

In some embodiments, for patches worn for 24 hours to 3 days, the matrix layer has a weight of 30 GSM (grams per square meter) to 100 GSM. For patches worn for 7 days, the matrix layer has a weight of 40 GSM to 150 GSM. For patches worn for 14 days, the matrix layer has a weight of 50 GSM to 700 GSM.

### Definitions

As used herein, the term “pharmaceutically acceptable salt” refers to those salts that are suitable for contact with subjects (e.g., human subjects) within reasonable medical judgment without excessive toxicity, irritation, allergic reaction, etc., have a reasonable benefit/risk ratio, and are effective for their intended use.

The “pharmaceutically acceptable salt” mentioned in the present invention include inorganic acid addition salts and organic acid addition salts, can be prepared in situ during the final separation and purification of compounds, or can be prepared by reacting a purified compound in the form of a free base (for example, risperidone) with an appropriate organic acid or inorganic acid separately and separating the salts thus formed. Examples of inorganic acid addition salts include, but are not limited to, sulfates, pyrosulfate, bisulfate, sulfite, bisulfite, nitrate, phosphate, monohydrogen phosphate, dihydrogen phosphate, metaphosphate, pyrophosphate, hydrochloride, hydrobromide, hydroiodide, phosphite, borate, etc. Examples of organic acid addition salts include saturated or unsaturated C₁-C₃₀ fatty acid salts, including but not limited to monocarboxylic acid salts or dicarboxylic acid salts. Non-limiting examples include formate, glyoxylate, oxalate, acetate, glycolate, acrylate, pyruvate, malonate, propionate, 3-hydroxypropionate, lactate, glycerate, fumarate, maleate, oxaloacetate, crotonate, acetoacetate, 2-oxobutyrate, methylmalonate, succinate, malate, L-tartrate, DL-tartrate, meso-tartrate, dihydroxytartrate, butyrate, isobutyrate, hydroxybutyrate, levulinate, sorbate, itaconate, mesaconate, ketoglutarate, glutarate, succinate, methyl succinate, valerate, isovalerate, pivalate, cis-aconitate, trans-aconitate, ascorbate, citrate, isocitrate, adipate, caproate, benzoate, salicylate, gentisate, protocatechuate, gallate, cyclohexane carboxylate, pimelate, benzoate, chlorobenzoate, phthalate, isophthalate, terephthalate, terephthalate, phenyl acetate, toluate, o-toluate, m-toluate, p-toluate, dinitrobenzoate, benzenesulfonate, tosylate, citrate, mesylate, oleate, tosylate, methanesulfonate, naphthoate, glucoheptonate, lactobionate, lauryl sulfonate and isethionate, mandelate, homogentisate, suberate, caprylate, decanoate, laurate, palmitate, stearate, isostearate, oleate, elaidate, gangduosuanyan [symbol] [symbol] , erucate, nervonate, and ximenic acid salt [symbol] , hexadecatrienoic acid salt, linoleate, α-linolenate, γ-linolenate, calendula acid salt, stearate, mead acid salt, eicosadienoic acid salt, eicosatrienoic acid salt, dihomo-γ-linolenate, arachidonic acid salt, docosadienoic acid salt, etc. and combinations thereof.

As used herein, the term “therapeutically effective amount” refers to the amount of a compound or molecule of the present invention that, when administered to a subject, (i) treats or prevents a specific disease, disorder or condition, (ii) attenuates, improves or eliminates one or more symptoms of the specific disease, disorder or condition, or (iii) prevents or delays the onset of one or more symptoms of the specific disease, disorder or condition described herein.

As used herein, the term “about” means plus or minus 10% of the indicated number. For example, “about 10%” may represent a range of 9% to 11%, and “about 1” may represent 0.9-1.1.

As used herein, the terms “optional” or “optionally” include both a selection and a non-selection. For example, “optional risperidone crystal form A” refers to the two situations of using risperidone crystal form A and not using risperidone crystal form A; “Optional other excipients” refers to two situations of using other excipients and not using other excipients.

As used herein, the term “treatment” refers to clinical intervention that attempts to alter the natural course of the individual being treated, and may be performed for prevention or in the course of clinical pathology. Desirable effects of treatment include, but are not limited to, preventing occurrence or recurrence of disease, alleviation of symptoms, weakening of any direct or indirect pathological consequences of the disease, preventing metastasis, decreasing the rate of disease progression, amelioration or alleviation of the disease state, and remission or improved prognosis.

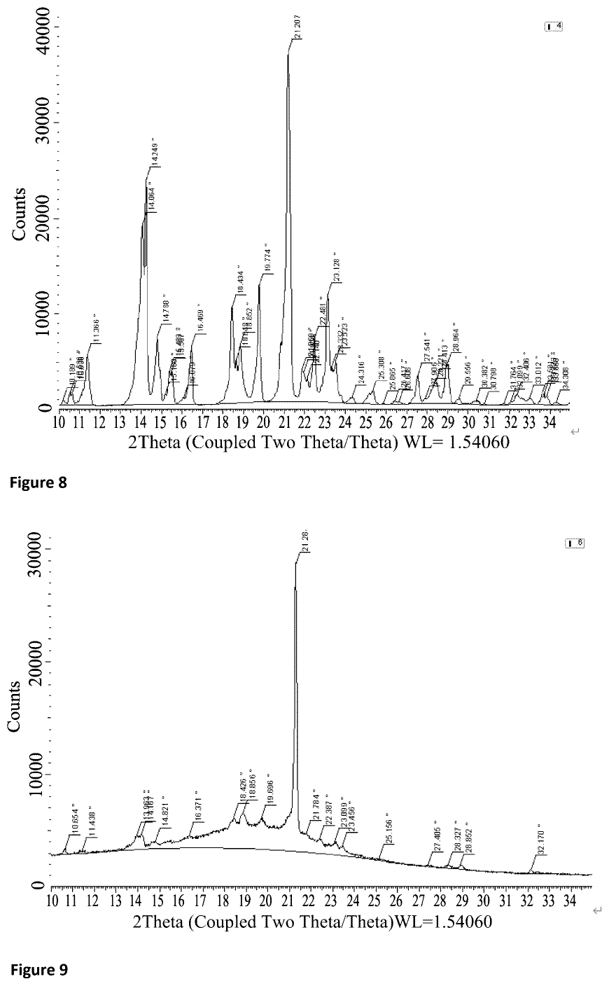

As used herein, the term “risperidone crystal form A” is described in WO 0212200A1, and its XRPD spectrum has characteristic peaks at 2θ values of 14.2±0.2°, 21.3±0.2°, and further has 2θ values of 10.6±0.2°, 11.4±0.2°, 16.4±0.2°, 18.9±0.2°, 19.9±0.2°, 22.5±0.2°, 23.3±0.2°, 25.4±0.2°, 27.6±0.2°, 29.0±0.2°. The risperidone crystal form A used in the present invention has an XRPD spectrum as shown in FIG. 8.

As used herein, the term “micronized” refers to particles having a particle size of 20 μm or less. In some embodiments, 90% or more of the particles have an average diameter of 20 μm or less, more preferably from 0.5 nm to 20 μm, and particularly preferably from 0.5 nm to 15 μm.

As used herein, the terms “backing layer” (in FIGS. 1, 2, 3) and separation layer (in FIGS. 4A-4F) serve as the upper surface of a transdermal patch and provide flexibility to the patch as main structural elements. Preferably, the backing layer is substantially impermeable to transdermally administered pharmaceutical compositions. The backing layer is preferably made from a sheet or film of flexible elastic material. The backing layer is preferably air-impermeable. The backing layer used in the patch of the present invention is preferably made of a flexible, biocompatible material that mimics the elastic properties of the skin and conforms to the skin during movement. Non-occlusive backing layers allow the area to breathe (i.e. promote water vapor transmission across the skin surface), whereas occlusive backing layers reduce air/vapor penetration. Preferably, the backing layer of the matrix-type transdermal administration systems (FIGS. 1-4) is occlusive. Preferably, the backing layer contains synthetic polymers such as polyolefins, polyesters, polyethylene, polyvinylidene chloride, polypropylene, polyethylene vinyl acetate and polyurethanes. Preferably, the thickness of the backing layer is from about 0.5 mil to about 5 mil; more preferably, the thickness of the backing layer is from about 1 mil to about 3 mils. preferably, the oxygen transmission rate is from about 2 cc/m/24 hr to about 100 cc/m/24 hr, and more preferably, the oxygen transmission rate is from about 70 g/m/24 hr to about 90 g/m/24 hr. Preferably, the MVTR is from about 0.1 g/m/24 hr to about 50 g/m/24 hr, more preferably, the MVTR is from about 0.3 g/m/24 hr to about 30 g/m/24 hr. In a preferred embodiment, the backing layer is an of occlusive polyester film layer of approximately 2.0 mil thick (commercially available as Scotchpak 9733, Scotchpak 9735 and Scotchpak 9723, 3 M Drug Delivery Systems, St. Paul Minn.). Scotchpak 9733 composed of polyester and medium density polyethylene/ethylene vinyl acetate heat-sealing layer, and the laminate is translucent, conformable, occlusive and heat-sealable. It can be used in the matrix-type transdermal administration system shown in FIGS. 1-3 and 4A-4F. More preferably, the backing layer includes a laminated material including an aluminum foil layer between polymer film layers, such as Scotchpak 9738 and Scotchpak 1109. When the patch is applied to the skin, the aluminum layer prevents light from coming into contact with the photosensitive risperidone.

As used herein, the term “separation layer” can be used to separate the overlapping adhesive film layer and the drug-containing matrix layer of the patch in FIGS. 4A-4F. The separation layer is preferably a nonwoven polyester layer. Specific separation layers can be selected from Scotchpak1109, Scotchpak 9738, Scotchpak 9733 and Scotchpak 9754.

As used herein, the term “composite film” refers to a composite having a multi-layer structure. For example, in the present invention, “release liner layer/drug matrix layer/backing layer composite film” and “backing layer/first overlapping adhesive film/second overlapping adhesive film/release film composite film” all have multi-layer composite structures. Unless otherwise specified, “/” is used in the present invention to divide different layers in the composite film.

As used herein, the term “matrix layer” contains risperidone dispersed in the matrix layer in crystalline and non-crystalline, a skin penetration enhancer and a pressure-sensitive adhesive and optional other excipients.

In addition, the matrix layer may also contain one or more other pharmaceutically acceptable excipients. Non-limiting examples of other excipients include, but are not limited to, cohesion promoters, antioxidants, anti-skin irritation agents, tackifiers, plasticizers, and the like.

Non-limiting examples of antioxidants include tocopherol, tocopheryl acetate, potassium metabisulfite, sodium metabisulfite, sodium bisulfite, sodium sulfite, propyl gallate, thioglycerol, sodium thiosulfate, sodium dioxide, sodium formaldehyde sulfoxylate, butylated hydroxytoluene (BHT), and chelating agents that act as synergistic antioxidants, including citric acid, tartaric acid, calcium disodium edetate, disodium edetate and EDTA. Preferably, the antioxidant is alpha-tocopherol (i.e. vitamin E), or a combination of alpha-tocopherol and butylated hydroxytoluene (BHT).

The pressure-sensitive adhesives include, but are not limited to, acrylic adhesives, organosilicon adhesives, acrylic-organosilicon copolymer adhesives, polybutylene adhesives, styrene-isoprene-butylene copolymers, styrene-butadiene-styrene copolymer, or a combination of two or more adhesives. Examples of acrylic adhesives are Duro-Tak® 387-2516, Duro-Tak®k 387-4280, Duro-Tak® 387-2287, Duro-Tak® 387-2510, Duro-Tak® 87-2196, Duro-Tak® 387-2051, Duro-Tak® 387-2052, Duro-Tak® 387-2054, Duro-Tak® 87-2194, Duro-Tak® 87-235A, Duro-Tak® 87-900A, Duro-Tak® 87-9301, Duro-Tak® 87-4098, GEFVA GMS® 788, GEFVA GMS® 9073, Duro-Tak® 387-2353, Duro-Tak® 87-2074, Duro-Tak® 87-2852, Duro-Tak® 87-2054, GELVA®-73 Tak® 80-1196, Duro-Tak® 87-2070, Duro-Tak® 87-2979, Duro-Tak® 87-2888 and Duro-Tak® 87-2296 from Henkel Corporation (Bridgewater, New Jersey). Organosilicons BIO-PSA® 7-4401, BIO-PSA® 7-4402, BIO-PSA® 7-4501, BIO-PSA® 7-4502, BIO-PSA® 7-4601, BIO-PSA® 7-4602, SRS7-4502, SRS7-4501, SRS7-4502, SRS7-4602, BIO-PSA® 7-4101, BIO-PSA® 7-4102, BIO-PSA® 7-4103, BIO-PSA® 7-4201, BIO-PSA® 7-4202, BIO-PSA® 7-4203, BIO-PSA® 7-4301, BIO-PSA® 7-4302, BIO-PSA® 7-4302, BIO-PSA @7-6101, BIO-PSA®7-6102, BIO-PSA @7-6201, BIO-PSA®7-6301, BIO-PSA®7-6302 from Dupont (Midland, MI). Examples of polybutylene adhesives are combinations of two or more low molecular weight polyisobutylenes (Oppanol B10, Oppanol B11, and Oppanol B12 from BASF), medium molecular weight polyisobutylene (Oppnaol B50, Oppanol B80, and Oppnaol N80), and high molecular weight polyisobutylene (Oppanol B100, Oppanol B150, Oppanol N100, and Oppanol N150). An example of a styrene-isoprene-butylene copolymer is D1161 (Kraton) and 5229, 5002 and 5505 (Eneos).

Preferably, the pressure-sensitive adhesive is an acrylic adhesive, more preferably a hydroxyethyl functional acrylic copolymeric adhesive, such as Duro-Tak 387-2516, Duro-Tak 387-4280, 387-2287, and Duro-Tak 387-2510. The amount of the adhesive is 50-82% relative to the total weight of the matrix layer; preferably, 50-80% or 55-80%. Preferably, the pressure-sensitive adhesive is a pressure-sensitive adhesive solution whose solvent is a poor solvent for risperidone. Examples of the solvents are n-heptane and ethyl acetate. n-Heptane is a poor solvent for risperidone, so most of the risperidone crystal form A is not dissolved in the pressure-sensitive adhesive solution, but exists in the pressure-sensitive adhesive blend as the original polymorph of the crystal. When the solvent is removed during the coating and drying process, risperidone is present in the dried drug matrix layer as the original stable polymorph A. Non-limiting examples of the tackifiers include polybutene, terpenes and mixtures thereof. The tackifier is present in the matrix layer in an amount from about 0% to about 30%, preferably from about 0% to about 25%, by weight of the adhesive material.

Non-limiting examples of the cohesion promoters are soluble polyvinylpyrrolidone (also known as povidone), insoluble crospovidone, Eudragit E, Eudragit EPO, Eudragit S, Eudragit R. Plastoid B, mixtures thereof, and the like. The matrix layer of the present invention contains a liquid skin penetration enhancer that causes cold flow around the patch, which can cause dark rings to appear on the skin during wear of the patch or transfer of adhesive to the skin when the patch is removed from the skin. Cohesion promoters increase the cohesion of the matrix layer, reducing cold flow, reducing the appearance of the dark ring on the skin or reducing the transfer of adhesive to the skin. An example of the soluble povidone is Povidone K90 and an example of the insoluble crospovidone is CL-M. The present inventors have surprisingly found that Povidone K90 is an excellent cohesion promoter. Preferred cohesion promotors are povidone K90, Eudragit EPO, and povidone K90 and Eudragit EPO, crospovidone CL-M and Eudragit EPO, povidone K90 and crospovidone CL-M and Eudragit EPO, povidone K90 and Plastoid B, crospovidone CL-M and Plastoid B, povidone K90 and crospovidone CL-M, and a preferred combination is one of povidone K90 and Eudragit EPO.

The skin penetration enhancers include, but are not limited to, C₁-C₃₀ fatty acids. In some embodiments this may specifically be C₇₋₃₀ fatty acid, C₇₋₂₂ fatty acid, or C₁₂₋₂₂ fatty acid. The C₁-C₃₀ fatty acids include saturated or unsaturated fatty acids, including but not limited to monocarboxylic acids or dicarboxylic acids. Non-limiting examples include formic acid, glyoxylic acid, oxalic acid, acetic acid, glycolic acid, acrylic acid, pyruvic acid, malonic acid, propionic acid, 3-hydroxypropionic acid, lactic acid, glyceric acid, fumaric acid, maleic acid, oxalacetic acid, crotonic acid, acetoacetic acid, 2-oxobutyric acid, methylmalonic acid, succinic acid, malic acid, L-tartaric acid, DL-tartaric acid, meso-tartaric acid, dihydroxytartaric acid, butyric acid, isobutyric acid, hydroxybutyric acid, levulinic acid, sorbic acid, itaconic acid, meconic acid, ketoglutaric acid, glutaric acid, methyl succinic acid, valeric acid, isovaleric acid, pivalic acid, cis-aconitic acid, trans-aconitic acid, ascorbic acid, citric acid, isocitric acid, adipic acid, caproic acid, benzoic acid, salicylic acid, gentisic acid, protocatechuic acid, gallic acid, cyclohexanecarboxylic acid, pimelic acid, phthalic acid, isophthalic acid, isophthalic acid, terephthalic acid, terephthalic acid, phenylacetic acid, toluic acid, o-toluic acid, m-toluic acid, p-toluic acid, mandelic acid, homogentisic acid, octanedioic acid, suberic acid, caprylic acid, capric acid, lauric acid, palmitic acid, stearic acid, isostearic acid, oleic acid, elaidic acid, gangduosuan [symbol] , erucic acid, nervonic acid, and simonic acid [symbol] , hexadecatrienoic acid, Linoleic acid, α-linolenic acid, γ-linolenic acid, calendula acid, stearic acid, mead acid, eicosadienoic acid, eicosatrienoic acid, dihomo-γ-linolenic acid, arachidonic acid, docosadienoic acid and combinations thereof. Preferred is oleic acid, isostearic acid, or stearic acid.

The skin penetration enhancers also include fatty alcohols. The fatty alcohols include but are not limited to one or more saturated, monounsaturated or polyunsaturated C₇₋₃₀ alkyl alcohol or C₃₋₃₀ alkenyl alcohol, preferably one or more saturated, monounsaturated or polyunsaturated C₁₃₋₃₀ alkyl alcohol or C₁₃₋₃₀ alkenyl alcohol. This may include, but is not limited to, one or more of the following: tridecanol, myristyl alcohol, pentadecyl alcohol, cetyl alcohol, palmitoleyl alcohol, heptadecanol, stearyl alcohol, oleyl alcohol, linoleyl alcohol, nonadecanol, arachidonoyl alcohol, octyldodecanol, behenyl alcohol, sinapyl alcohol, lignoceryl alcohol and lignoceryl alcohol. Saturated fatty alcohol penetration enhancers may include but are not limited to one or more of the following: myristyl alcohol, isomyristyl alcohol, trans-isomyristyl alcohol, cetyl alcohol, isopalmityl alcohol, trans-isopalmityl alcohol, stearyl alcohol, isostearyl alcohol and trans-isostearyl alcohol. In some embodiments, the fatty alcohol is myristyl alcohol.

The skin penetration enhancers also include fatty esters. The fatty esters are esters produced by C₁-C₃₀ fatty acids and C₁-C₃₀ alcohols in combination. Non-limiting examples are propylene glycol mono laurate, lauryl lactate, myristyl lactate, cetyl lactate, palmitoyl lactate, Ceraphyl 31, ethyl laurate, methyl laurate, isopropyl myristate, isopropyl palmitate, Diisoadipate, medium chain fatty acid triglycerides, diethyl sebacate.

The skin penetration enhancers also include surfactants. Surfactants include, but are not limited to, one or more of glycerides (monoglycerides, diacylglycerol, and triglyceride), polyoxyethylene stearate, a mixture of trioctatetraenyl-4 phosphate, ethylene glycol palmityl stearate and diethylene glycol palmityl stearate, polyglycerol-3 diisostearate, PEG-6 stearate and ethylene glycol palmityl stearate and PEG-32 stearate, oleyl polyoxy-6 glyceride, lauroyl polyoxy-6 glyceride, capryloyl polyoxy-8 glyceride, propylene glycol monocaprylate, propylene glycol monolaurate, propylene glycol monocaprylate, Polyglyceryl-3 dioleate, a mixture of PEG-6 stearate and PEG-32 stearate, lecithin, cetyl alcohol, cholesterol, dioctyl sodium sulfosuccinate, sodium dodecyl sulfate, triethanolamine stearate, polyoxyethylene fatty alcohol ether, glyceryl monostearate, sorbitan monolaurate, lanolin alcohol and ethoxylated lanolin alcohol, sorbitan fatty acid ester, sucrose distearate. Span 20, Span 40, Span 80, Tween 20 Tween 40 Tween 80 pentadecanolide, glyceryl monolactate, glyceryl monostearate, glyceryl monooleate or combinations thereof.

According to a preferred embodiment of the present invention, the weight percentage of the skin penetration enhancer is 1-50% of the drug reservoir layer, preferably 2-30%.

Optional non-limiting solubilizers or solvents include C₁-C₆ alkyl alcohols, n-heptane, ethyl acetate, toluene, or a combination of two or more thereof. Solubilizers or solvents can also reduce the viscosity of the drug-adhesive suspension so that the suspension can be spread into a uniform film.

As used herein, the term “semipermeable membrane or machine-woven fabric layer” is used to contain a liquid or semi-solid matrix material within the matrix drug layer, and has a function of controlling the diffusion of risperidone from the liquid or semi-solid matrix drug layer to the skin-contacting adhesive layer. The semipermeable membrane includes, but is not limited to, ethylene-co-vinyl acetate copolymer membranes, polyethylene polymer membranes, and polypropylene polymer membranes. Non-limiting examples of ethylene-co-vinyl acetate copolymers include 3M Cotran 9702, Cotran 9712, Contan 9716 and Contran 9728. Non-limiting examples of polyethylene polymer membranes include Solupore. Non-limiting examples of polypropylene polymer membranes include Celgard 2400. The semipermeable membrane or machine-woven fabric layer and backing layer can be sealed together around the peripheral edge.

Suitable semipermeable membranes include continuous membranes and microporous membranes and can be made from woven or non-woven materials. The semipermeable membrane is preferably made of a flexible polymeric material commonly used by those skilled in the art. Polymer membranes that may be used to make the semipermeable membrane layer include, but are not limited to, those containing low density polyethylene, high density polyethylene, ethyl vinyl acetate copolymer, polypropylene, and other suitable polymers. In one embodiment, the semipermeable membrane layer is a microporous membrane prepared from an ethylene-vinyl acetate copolymer containing from about 0.5 to about 28 wt. % vinyl acetate. Suitable woven materials include Saatifil PES, such as PES 105/52 available from Saatitech, Inc. A suitable non-woven fabric is Sontara from DuPont Nonwovens Sontara Technologies. In a preferred embodiment, the semipermeable membrane layer is an ethylene-vinyl acetate copolymer membrane available from 3M™, such as Cotran 9702, Cotran 9705, Cotran 9706, Cotran 9707, Cotran 9712, Cotran 9715, Cotran 9716 and Cotran 9728 (available from 3M™). The thickness of the semipermeable membrane layer may generally be from about 10 μm to about 100 μm, preferably from about 15 μm to about 50 μm.

As used herein, the term “skin-contacting adhesive layer” serves to adhere the risperidone transdermal administration system to the skin surface. It can also be used to control the rate of risperidone delivery to the skin after the protective release liner layer is removed. The skin-contacting adhesive layer includes a pressure-sensitive adhesive and optional risperidone and optional other excipients selected from penetration enhancers, cohesion promoters, antioxidants, anti-skin irritation agents, tackifiers, plasticizers, solubilizers, and solvents. The pressure-sensitive adhesive includes one or more of acrylic adhesive, methacrylic adhesive, polyisobutylene adhesive, styrene-isoprene-styrene block copolymer adhesive, siloxane adhesive, acrylic-copolysiloxane copolymer adhesive; The acrylic adhesive is selected from Henkel's Duro-Tak adhesive 387-2051, 387-2054, 387-2353, 87-235A, 87-2852, 87-2074, 87-2677, 387-2516, 387-2287, 387-4287, 387-2510, crosslinked 387-2510, 87-900A, 87-9301, 87-4098, 87-2194, Gelva GMS788, Gelva GMS 9073, Gelva 737, Gelva 2655, Polythick 410-SA (Sanyo Chemical Industry Co., Ltd.); The polyisobutylene adhesive is selected from Oppanol N150, Oppanol B150, Oppanol N100, Oppnaol B100, Oppanol N80, Oppanol B80, Oppanol B10, B11, B12 and low molecular weight polybutylene H1900 from Ineos and mineral oil tackifier; The siloxane adhesive is selected from DuPont Bio-PSA 7-4100, 7-4200, 7-4300, 7-4400 and 7-4500, 7-4600 Bio-PSA SR7-4400, SRS7-4500, and SRS7-4600; The acrylic-co-polysiloxane copolymer adhesive is selected from DuPont Bio-PSA 7-6100, 7-6200 and 7-6300. The styrene-isoprene-styrene block copolymer adhesives are selected from D1161 from Kraton, and 5229, 5002 and 5502 from Eneos. Acrylic adhesives combined with organosilicon adhesives or polyisobutylene and polyisobutylene combined with styrene-isoprene-styrene are also good adhesive choices.

As used herein, the term “overlapping adhesive film” refers to a patch comprising a backing layer-overlapping adhesive film-separation layer-matrix layer-release liner layer composed of an elastic material (as shown in FIGS. 4A-4F, FIG. 13), which adheres to the skin more firmly than a patch comprising a backing layer-drug matrix layer-release liner layer (as shown in FIG. 1). The overlapping adhesive film can be one layer, two layers or multiple layers. The transdermal administration system of two layers of overlapping adhesive films is shown in FIGS. 4B, 4D and 4E. The overlapping adhesive film layer 1 can prevent the overlapping adhesive film layer 2 from migrating to the side of the backing layer away from the skin. The overlapping adhesive films contain adhesives, examples of which include Duro-Tak 387-2516, Duro-Tak 387-2287, Duro-Tak 387-4287, Duro-Tak 87-2051, Duro-Tak 87-2052, Duro-Tak 87-2054 and other acrylates, siloxane, polyisobutylene, styrene-isoprene-styrene copolymer, or a combination of two or more adhesives. The present invention found that when the overlapping adhesive film is one or two acrylates, a considerable amount of nonvolatile risperidone migrates from the drug-containing matrix layer to the overlapping adhesive film layer, but when the overlapping adhesive film layer connected with the separation layer is siloxane, polyisobutylene or styrene-isoprene-styrene copolymer, only a small amount of nonvolatile risperidone migrates from the drug-containing matrix layer to the overlapping adhesive film layer.

As used herein, the term “release liner layer” includes, but is not limited to, silicone-coated polyester release liner available from many suppliers, fluoropolymer-coated polyester release liner from 3 M, and fluorosilicone-coated polyester release liner.

### The Present Invention has the Advantages and Technical Effects as Follows

The prior art does not disclose that a transdermal administration system containing risperidone or a pharmaceutically acceptable salt thereof can continuously release drugs to patients in a controlled manner at a constant rate, and not disclose a method for continuously delivering risperidone or a pharmaceutically acceptable salt thereof at a constant rate at a therapeutically effective amount of blood drug concentration within 24 hours to 14 days.

1. According to the invention, the risperidone crystal form A or a pharmaceutically acceptable salt thereof is combined with a skin penetration enhancer, so that the risperidone crystal form A has good stability, and the risperidone or a pharmaceutically acceptable salt thereof can be continuously delivered to permeate the skin into the blood circulation system. Unexpectedly, the transdermal administration system of the present invention enables sustained delivery of risperidone or a pharmaceutically acceptable salt thereof at a therapeutically effective amount of blood drug concentration within 24 hours to 14 days.

2. According to the invention, the skin flux can be increased by controlling the ratio of the skin penetration enhancer and risperidone crystal form A, which is beneficial to achieving the above technical effects.

3. According to the invention, the skin flux can be increased by including undissolved risperidone crystal form A in the matrix layer of the risperidone administration system of the present invention, which is beneficial to achieving the above technical effects.

4. According to the invention, the viscosity of the transdermal administration system can be improved by adjusting the amounts of the pressure-sensitive adhesive and the cohesion promoter relative to the total weight of the matrix layer, so that the system can be well tolerated by the skin.

Therefore, the present invention obtains a stable risperidone transdermal administration system, which can continuously release the drug to patients in a rate-controlled manner, and continuously deliver risperidone or a pharmaceutically acceptable salt thereof at a constant rate at a therapeutically effective amount of blood drug concentration within 24 hours to 14 days. The risperidone transdermal administration system of the present invention has stable properties, is safe and non-irritating, has good adhesion and stickiness to the skin, and has good skin tolerance.

5. The present invention increases the stickiness of the patch to the skin through the use of overlapping adhesive film layers.

6. The present invention uses a different adhesive from the central drug-containing layer (i.e. matrix layer) in the overlapping adhesive film, thereby greatly reducing the migration of non-volatile component drug molecules from the drug-containing layer to the overlapping adhesive film layer.

7. In the present invention, the separation layer and the drug-containing layer are cut into small pieces of 2-20 cm² to increase the stickiness of the drug-containing layer to the skin.

8. The separation layer pieces and drug-containing layer of the present invention are partially connected to each other. This makes the die-cutting process easier than that without the partial connection. Small pieces with no partially connected become messy during the die-cutting process. The small pieces of the separation drug layers that are partially connected to each other are neatly organized during the die-cutting process.

### EXAMPLES

In order to make the objectives, technical solutions and advantages of the embodiment of the present invention more clear, the technical solutions in the embodiment of the present invention will be described clearly and completely below. Apparently, the described embodiment is a module embodiment of the present invention, but not all of the embodiments. Elements and features described in one embodiment of the present invention may be combined with elements and features illustrated in one or more other embodiments. It should be noted that for purposes of clarity, representation and description of components and processes known to those of ordinary skill in the art that are not relevant to the present invention have been omitted from the description. Based on the embodiments of the present invention, all the other embodiments obtained by those of ordinary skill in the art without any inventive efforts should fall within the scope of protection of the present invention.

### In Vitro Skin Flux Experiment:

In vitro penetration testing was performed using a vertical statically modified Franz cell. The receptor chamber has a volume of 7 ml, is filled with a buffer solution at pH 7.0, and has an effective skin permeability of 0.61 cm². The human cadaver skin was mounted on the receptor chamber with the dermis side facing the receptor chamber. The matrix layer was placed on the stratum corneum side of the human cadaver skin. The O-ring was placed on top of the skin. The donor chamber was secured on top of the receptor chamber. The Franz cell was placed in a 32° C. incubator on a magnetic stir plate. At each prearranged time point, 2 ml of solution was taken, the remaining solution was poured off, and refilled with new receptor fluid. The receptor fluid was immediately analyzed for the amount of risperidone by HPLC.

### Comparative Examples 1 to 6

K 90, oleic acid, EPO, DL-alpha tocopherol, 0.002% ascorbyl palmitate NF, 0.1% butylated hydroxytoluene and risperidone were dissolved in ethanol. Crospovidone was added and mixed for 24 hours. Duro-Tak 387-2516 was added and mixed well. The wet blend was coated on a release film, dried, and then laminated to a backing layer Scotchpak 1109. Comparative examples 5 to 6 were prepared in a similar manner to comparative example 1. An in vitro skin flux test was performed and the results are shown in Table 1 and FIG. 5. The in vitro skin flux was low. From comparative example 1 to comparative example 3, under 3% risperidone, when oleic acid increased from 10% to 12% to 17%, the in vitro skin flux decreased. This was because when the solubilizer oleic acid was increased, the drug concentration in the oleic acid decreased and became unsaturated. Comparative examples 4 to 6 also had the same trend.

TABLE 1
Skin flux of comparative example 1 to comparative example 6

| | Average value of skin from two donors (S365 & S376) | | | | | |
|---|---|---|---|---|---|---|
| | Comparative | Comparative | | | | |
| | example 1 | example 2 | Comparative | | | |
| | Comparative | Comparative | example 3 | Comparative | Comparative | Comparative |
| | preparation | preparation | Comparative | example 4 | example 5 | example 6 |
| | 1 | 2 | preparation 3 | Comparative | Comparative | Comparative |
| | 3.0% | 3.0% | 3% | preparation 4 | preparation 5 | preparation 6 |
| | risperidone, | risperidone, | risperidone, | 3.5% | 3.5% | 3.5% |
| | 12% K 90, | 12% K 90, | 12% K 90, | risperidone, | risperidone, | risperidone, |
| | 12% oleic | 15% oleic | 17% oleic | 12% K 90, 10% | 12% K 90, 12% | 12% K 90, 15% |
| | acid, 0.5% | acid, 1.25% | acid, 1% EPO, | oleic acid, 1% | oleic acid, 1% | oleic acid, |
| | EPO, 0.1% | EPO, 0.1% | 0.1% DL- | EPO, 0.1% DL- | EPO, 0.1% DL- | 1.25% EPO, |
| | DL-alpha | DL-alpha | alpha | alpha | alpha | 0.1% DL-alpha |
| | tocopherol, | tocopherol, | tocopherol, | tocopherol, | tocopherol, | tocopherol, |
| | 0.002% | 0.002% | 0.002% | 0.002% | 0.002% | 0.002% |
| | ascorbyl | ascorbyl | ascorbyl | ascorbyl | ascorbyl | ascorbyl |
| | palmitate NF, | palmitate NF, | palmitate NF, | palmitate NF, | palmitate NF, | palmitate NF, |
| | 0.1% | 0.1% | 0.1% | 0.1% | 0.1% | 0.1% |
| | butylhydroxy- | butylhydroxy- | butylhydroxy- | butylhydroxy- | butylhydroxy- | butylhydroxy- |
| | toluene, 10% | toluene, 10% | toluene, 10% | toluene, 10% | toluene, 10% | toluene, 10% |
| | crospovidone, | crospovidone, | crospovidone, | crospovidone, | crospovidone, | crospovidone, |
| | 62.2980% | 58.5480% | 56.7980% | 63.2980% | 61.2980% | 58.0480% |
| Time | Duro-Tak | Duro-Tak | Duro-Tak | Duro-Tak 387- | Duro-Tak 387- | Duro-Tak 387- |
| (hours) | 387-2516 | 387-2516 | 387-2516 | 2516 | 2516 | 2516 |
| 0 | 0.000 | 0.000 | 0.000 | 0.000 | 0.000 | 0.000 |
| 4 | 0.015 | 0.031 | 0.018 | 0.019 | 0.000 | 0.000 |
| 8 | 0.000 | 0.000 | 0.000 | 0.024 | 0.000 | 0.000 |
| 10 | 0.000 | 0.000 | 0.000 | 0.032 | 0.000 | 0.000 |
| 24 | 0.013 | 0.011 | 0.000 | 0.304 | 0.257 | 0.059 |
| 32 | 0.136 | 0.121 | 0.080 | 0.764 | 0.653 | 0.253 |
| 48 | 0.293 | 0.270 | 0.166 | 1.142 | 1.031 | 0.439 |
| 56 | 0.553 | 0.425 | 0.281 | 1.535 | 1.381 | 0.679 |
| 72 | 0.623 | 0.515 | 0.340 | 1.613 | 1.474 | 0.727 |
| 80 | 0.792 | 0.684 | 0.451 | 1.873 | 1.709 | 0.931 |
| 96 | 0.832 | 0.678 | 0.468 | 1.878 | 1.711 | 0.958 |
| 104 | 0.944 | 0.783 | 0.553 | 2.068 | 1.834 | 1.076 |
| 120 | 0.915 | 0.748 | 0.540 | 1.888 | 1.755 | 1.021 |
| 128 | 1.065 | 0.882 | 0.641 | 2.063 | 2.002 | 1.204 |
| 144 | 1.000 | 0.801 | 0.622 | 1.891 | 1.833 | 1.128 |
| 152 | 1.169 | 0.975 | 0.758 | 2.162 | 2.010 | 1.265 |
| 168 | 1.117 | 0.895 | 0.718 | 2.057 | 1.913 | 1.216 |

### Example 1 and Example 3

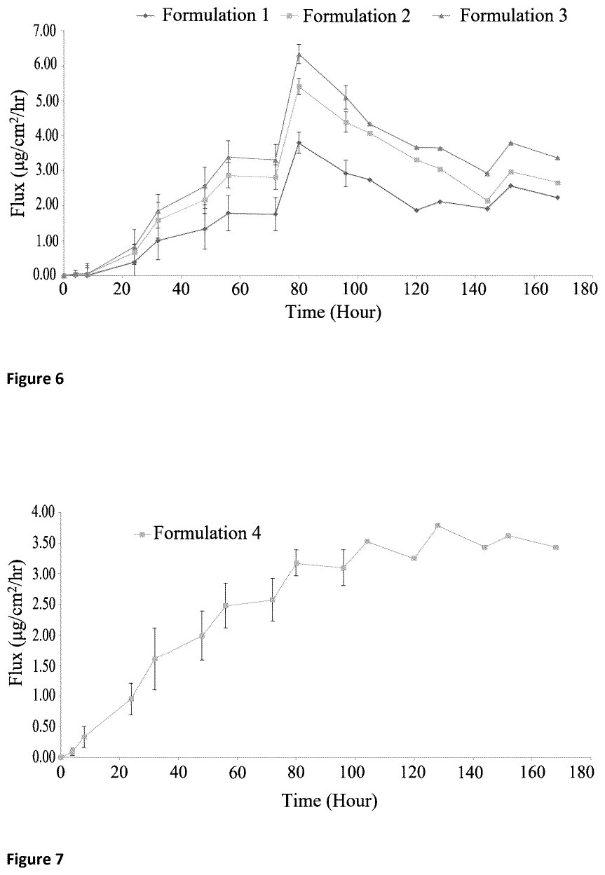

Examples 1 to 3 were prepared in a similar manner to comparative example 1. Some dissolved drugs recrystallized upon removal of the solvent. When the solubilizer oleic acid was fixed at 10%, the risperidone concentration increased from 4.1772% to 4.9964% to 5.7904%. An in vitro skin flux test was performed and the results are shown in Table 2 and FIG. 6. The in vitro skin flux increased. This was because the concentration of the drug in oleic acid increased. However, due to the low initial drug saturation and sub-saturation of the drug after 80 hours, the skin flux decreased after 80 hours.

TABLE 2
Skin flux for examples 1 to 3

| | Average value of skin from two donors | | |
|---|---|---|---|
| | Example 1 | Example 2 | Example 3 |
| | Preparation 1 | Preparation 2 | Preparation 3 |
| | 4.1772% risperidone, 10% | 4.9964% risperidone, 10% | 5.7904% risperidone, 10% |
| | oleic acid, 10% | oleic acid, 10% | oleic acid, 10% |
| | crospovidone, 0.1% DL- | crospovidone, 0.1% DL- | crospovidone, 0.1% DL- |
| | alpha tocopherol, 0.002% | alpha tocopherol, 0.002% | alpha tocopherol, 0.002% |
| | ascorbyl palmitate NF, 0.1% | ascorbyl palmitate NF, 0.1% | ascorbyl palmitate NF, 0.1% |
| | butylhydroxytoluene, | butylhydroxytoluene, | butylhydroxytoluene, |
| | 37.8104% Dow Corning 7- | 37.4008% Dow Corning 7- | 37.0038% Dow Corning 7- |
| Time | 6101, 37.8104% Dow | 6101, 37.4008% Dow | 6101, 37.0038% Dow |
| (hours) | Corning 7-6301 | Corning 7-6301 | Corning 7-6301 |
| 0 | 0.000 | 0.000 | 0.000 |
| 4 | 0.000 | 0.046 | 0.051 |
| 8 | 0.000 | 0.043 | 0.048 |
| 24 | 0.378 | 0.660 | 0.819 |
| 32 | 1.004 | 1.585 | 1.846 |
| 48 | 1.338 | 2.172 | 2.559 |
| 56 | 1.784 | 2.862 | 3.387 |
| 72 | 1.755 | 2.806 | 3.300 |
| 80 | 3.794 | 5.398 | 6.337 |
| 96 | 2.916 | 4.386 | 5.089 |
| 104 | 2.738 | 4.075 | 4.337 |
| 120 | 1.869 | 3.306 | 3.669 |
| 128 | 2.113 | 3.037 | 3.650 |
| 144 | 1.913 | 2.144 | 2.919 |
| 152 | 2.561 | 2.963 | 3.802 |
| 168 | 2.226 | 2.662 | 3.363 |

### Example 4

Example 4 was prepared in a similar manner to comparative example 1. Some dissolved drugs recrystallize upon removal of the solvent. An in vitro skin flux test was performed, and the results are shown in Table 3. The in vitro skin flux was very high and did not decrease within 168 hours. This was because the saturation of the 7.5% concentration was very high.

TABLE 3
Skin flux of example 4

| | Average value of skin from two donors |
|---|---|
| | 7.5% risperidone, 10% oleic acid, |
| | 20% crospovidone CL-M, 0.0583% DL-alpha tocopherol, |
| | 0.0167% ascorbyl palmitate NF, 0.1% |
| Time | butylhydroxytoluene, 21.8138% light mineral oil, |
| (hours) | 24.3068% Oppanol 12 Solution, 16.2045% Oppanol 100 |
| 0 | 0.000 |
| 4 | 0.093 |
| 8 | 0.338 |
| 24 | 0.957 |
| 32 | 1.604 |
| 48 | 1.987 |
| 56 | 2.476 |
| 72 | 2.572 |
| 80 | 3.171 |
| 96 | 3.097 |
| 104 | 3.522 |
| 120 | 3.247 |
| 128 | 3.779 |
| 144 | 3.433 |
| 152 | 3.617 |
| 168 | 3.429 |

### Example 5 to Example 8

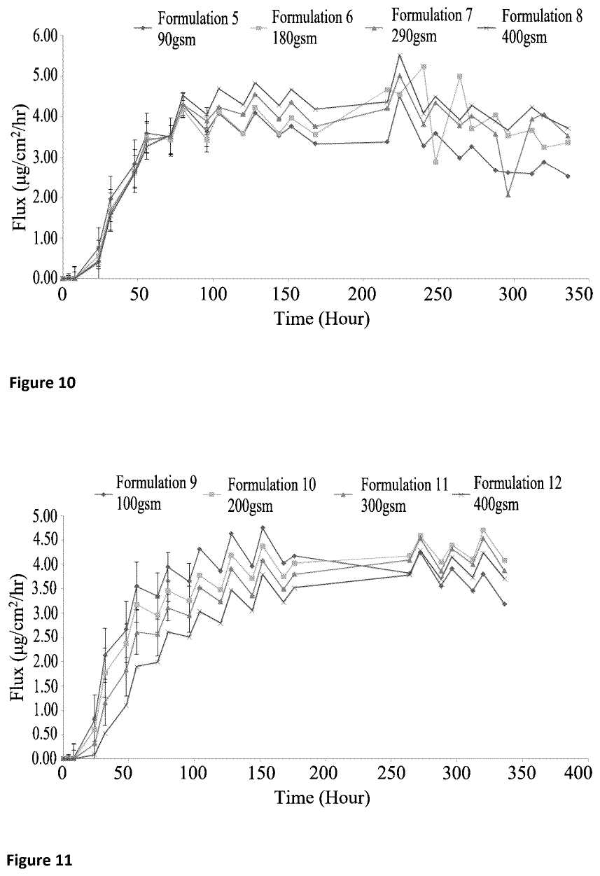

K 90, oleic acid, EPO, DL-alpha tocopherol, 0.002% ascorbyl palmitate NF, 0.1% butylated hydroxytoluene were dissolved in isopropanol. The solution was mixed for 24 hours. Duro-Tak 387-2516 was added and mixed well. Micronized risperidone crystal form A (FIG. 8, XRPD of API crystals) was added to form a homogeneous suspension in which undissolved risperidone crystals were suspended. The wet blend was coated on a release film, dried, and then laminated to a backing film Scotchpak 1109. Laminated material of 4 different coating weights (90 gsm, 180 gsm, 290 gsm and 400 gsm) were prepared. Microscopic analysis revealed that the laminated material was filled with risperidone crystals. The XRPD results (FIG. 9) indicate that the risperidone crystals have crystal form A. As shown in Table 4 and FIG. 10, the flux remains constant for 248 hours (10.5 days) with a coating weight of 90 gsm. When the coating weight was increased to 180 gsm, 290 gsm and 400 gsm respectively, the flux remains almost unchanged for 336 hours (14 days).

TABLE 4
Skin flux of example 5 to example 8

| | Average value of skin from two donors | | | |
|---|---|---|---|---|
| | Example 5 | Example 6 | Example 7 | Example 8 |
| | Preparation 5 | Preparation 6 | Preparation 7 | Preparation 8 |
| | 12% risperidone, | 12% risperidone, | 12% risperidone, | 12% risperidone, |
| | 10% oleic acid, 10% | 10% oleic acid, 10% | 10% oleic acid, 10% | 10% oleic acid, |
| | crospovidone, 0.1% | crospovidone, 0.1% | crospovidone, 0.1% | 10% crospovidone, 0.1% |
| | DL-alpha tocopherol, | DL-alpha tocopherol, | DL-alpha tocopherol, | DL-alpha tocopherol, |
| | 0.002% ascorbyl | 0.002% ascorbyl | 0.002% ascorbyl | 0.002% ascorbyl |
| | palmitate NF, 0.1% | palmitate NF, 0.1% | palmitate NF, 0.1% | palmitate NF, 0.1% |
| | butylhydroxytoluene, | butylhydroxytoluene, | butylhydroxytoluene, | butylhydroxytoluene, |
| time | 67.7980% Duro-Tak | 67.7980% Duro-Tak | 67.7980% Duro-Tak | 67.7980% Duro-Tak |
| (hours) | 387-2516, 90 gsm | 387-2516, 180 gsm | 387-2516, 290 gsm | 387-2516, 400 gsm |
| 0 | 0.000 | 0.000 | 0.000 | 0.000 |
| 4 | 0.000 | 0.011 | 0.030 | 0.000 |
| 8 | 0.000 | 0.000 | 0.000 | 0.000 |
| 24 | 0.748 | 0.555 | 0.444 | 0.395 |
| 32 | 1.968 | 1.699 | 1.637 | 1.548 |
| 48 | 2.839 | 2.638 | 2.653 | 2.600 |
| 56 | 3.584 | 3.474 | 3.417 | 3.261 |
| 72 | 3.497 | 3.426 | 3.514 | 3.546 |
| 80 | 4.274 | 4.176 | 4.293 | 4.524 |
| 96 | 3.633 | 3.422 | 3.886 | 4.041 |
| 104 | 4.075 | 4.116 | 4.226 | 4.682 |
| 120 | 3.561 | 3.586 | 4.051 | 4.284 |
| 128 | 4.087 | 4.217 | 4.546 | 4.822 |
| 144 | 3.528 | 3.586 | 3.938 | 4.272 |
| 152 | 3.754 | 3.964 | 4.341 | 4.658 |
| 168 | 3.329 | 3.556 | 3.755 | 4.174 |
| 216 | 3.375 | 2.879 | 4.193 | 4.350 |
| 224 | 4.500 | 4.993 | 5.014 | 5.513 |
| 240 | 3.269 | 3.698 | 3.807 | 4.070 |
| 248 | 3.587 | 4.044 | 4.337 | 4.486 |
| 264 | 2.972 | 3.522 | 3.772 | 3.913 |
| 272 | 3.264 | 3.661 | 4.010 | 4.270 |
| 288 | 2.669 | 3.243 | 3.569 | 3.877 |
| 296 | 2.618 | 3.359 | 2.062 | 3.657 |
| 312 | 2.585 | 3.226 | 3.935 | 4.226 |
| 320 | 2.883 | 3.622 | 4.059 | 4.009 |
| 336 | 2.523 | 3.162 | 3.519 | 3.711 |

### Example 9 to Example 12

K 90, oleic acid, EPO, DL-alpha tocopherol, 0.002% ascorbyl palmitate NF, 0.1% butylated hydroxytoluene were dissolved in isopropanol. The solution was mixed for 24 hours. Duro-Tak 387-2287 was added and mixed well. Micronized risperidone crystal form A was added to form a homogeneous suspension in which undissolved risperidone crystals are suspended. The wet blend was coated on a release film, dried, and then laminated to a backing film Scotchpak 1109. Laminated material of 4 different coating weights (90 gsm, 180 gsm, 290 gsm and 400 gsm) were prepared. Microscopic analysis showed that the laminated plate was filled with crystals. XRPD results indicate that the crystals have crystal form A. As shown in Table 4 and FIG. 11, the flux remains constant for 272 hours (11.5 days) with a coating weight of 100 gsm. When the coating weight was increased to 200 gsm, 300 gsm and 400 gsm respectively, the flux remains almost unchanged for 336 hours (14 days).

TABLE 5
Skin flux of example 9 to example 12

| | Average value of skin from three donors | | | |
|---|---|---|---|---|
| | Example 9 | Example 10 | Example 11 | Example 12 |
| | Preparation 9 | Preparation 10 | Preparation 11 | Preparation 12 |
| | 12% risperidone, 10% | 12% risperidone, 10% | 12% risperidone, 10% | 12% risperidone, 10% |
| | K90, 10% oleic acid, | K90, 10% oleic acid, | K90, 10% oleic acid, | K90, 10% oleic acid, |
| | 10% crospovidone, | 10% crospovidone, | 10% crospovidone, | 10% crospovidone, |
| | 1.25% EPO, 0.1% | 1.25% EPO, 0.1% | 1.25% EPO, 0.1% | 1.25% EPO, 0.1% |
| | DL-alpha tocopherol, | DL-alpha tocopherol, | DL-alpha tocopherol, | DL-alpha tocopherol, |
| | 0.002% ascorbyl | 0.002% ascorbyl | 0.002% ascorbyl | 0.002% ascorbyl |
| | palmitate NF, 0.1% | palmitate NF, 0.1% | palmitate NF, 0.1% | palmitate NF, 0.1% |
| | butylhydroxytoluene, | butylhydroxytoluene, | butylhydroxytoluene, | butylhydroxytoluene, |
| time | 56.5480% Duro- | 56.5480% Duro-Tak | 56.5480% Duro-Tak | 56.5480% Duro-Tak |
| (hours) | Tak387-2287, 100 gsm | 387-2287, 200 gsm | 387-2287, 300 gsm | 387-2287, 400 gsm |
| 0 | 0.000 | 0.000 | 0.000 | 0.000 |
| 4 | 0.000 | 0.000 | 0.000 | 0.000 |
| 8 | 0.017 | 0.000 | 0.000 | 0.000 |
| 24 | 0.810 | 0.607 | 0.308 | 0.088 |
| 32 | 2.128 | 1.768 | 1.162 | 0.524 |
| 48 | 2.665 | 2.378 | 1.830 | 1.110 |
| 56 | 3.547 | 3.170 | 2.609 | 1.895 |
| 72 | 3.345 | 2.960 | 2.563 | 1.988 |
| 80 | 3.939 | 3.456 | 3.106 | 2.612 |
| 96 | 3.643 | 3.251 | 2.947 | 2.508 |
| 104 | 4.315 | 3.767 | 3.524 | 3.033 |
| 120 | 3.853 | 3.473 | 3.229 | 2.790 |
| 128 | 4.627 | 4.179 | 3.905 | 3.466 |
| 144 | 3.958 | 3.709 | 3.358 | 3.050 |
| 152 | 4.750 | 4.376 | 4.074 | 3.787 |
| 168 | 4.020 | 3.742 | 3.488 | 3.221 |
| 176 | 4.164 | 4.021 | 3.792 | 3.516 |
| 264 | 3.811 | 4.173 | 4.079 | 3.778 |
| 272 | 4.240 | 4.591 | 4.534 | 4.286 |
| 288 | 3.553 | 4.045 | 3.856 | 3.690 |
| 296 | 3.903 | 4.397 | 4.315 | 4.139 |
| 312 | 3.457 | 4.102 | 3.993 | 3.724 |
| 320 | 3.801 | 4.706 | 4.532 | 4.228 |
| 336 | 3.183 | 4.083 | 3.866 | 3.697 |

### Physical Properties and Skin Adhesion-Finger Stickiness Results

The patch was placed on the workbench with the adhesive layer facing up. The adhesive was pressed with index finger for 5 seconds, then the index finger was lifted while holding part of the patch with the fingers of the other hand. The thumb was used to touch the front of the index finger to observe if there was any stickiness on the finger. If the adhesive was transferred to the index finger after the adhesive layer of the patch was lifted from the adhesive layer, the finger is sticky. The softer the adhesive, the less cohesive it is in terms of rheology, and the more adhesive transfer can be expected from the patch adhesive to the finger. Adhesive transfer to the skin can reduce skin adhesion and increase dark rings around the perimeter of the patch.

The results are shown in Table 6. The commercial patch was used as a reference for finger stickiness and was rated 10. It had no adhesive transfer to the fingers. In Example 13, preparation 13 had a finger stickiness of 15 and the adhesive transferred to 40% of the touching fingers. In examples 14 to 23, different levels of crospovidone CL-M, povidone K90 and Eudragit EPO were used to obtain preparations with finger stickiness in the range of 1 to 15 and no adhesive transfer to the contact fingers, except for preparation 22, because the amount of Eudragit was insufficient.

TABLE 6
Finger stickiness results

| | | | | | 168-hour skin |
|---|---|---|---|---|---|
| | | | | Amount of | adhesion |
| | | | | adhesive | score (0-4 |
| | | | Finger | transferred | scoring |
| Example# | Preparation# | Preparation composition | stickiness | to fingers | system) |
| 13 | Commercial | Commercial patch | 10 | 0 | Not carried |
| | patch | | | | out |
| 14 | 14 | 10% risperidone, 10% oleic acid, 0% | 15 | 40% | Not carried |
| | | crospovidone, 0.1% DL-alpha | | | out |
| | | tocopherol, 0.002% ascorbyl | | | |
| | | palmitate NF, 0.1% | | | |
| | | butylhydroxytoluene, 79.7980% | | | |
| | | Duro-Tak 387-2516 | | | |
| 15 | 15 | 10% risperidone, 10% oleic acid, 10% | 14 | 0 | 2 |
| | | crospovidone, 0.1% DL-alpha | | | |
| | | tocopherol, 0.002% ascorbyl | | | |
| | | palmitate NF, 0.1% | | | |
| | | butylhydroxytoluene, 69.7980% | | | |
| | | Duro-Tak 387-2516 | | | |
| 16 | 16 | 11% risperidone, 15% K90, 10% oleic | 13 | 0 | Not carried |
| | | acid, 0% EPO, 0.1% DL-alpha | | | out |
| | | tocopherol, 0.002% ascorbyl | | | |
| | | palmitate NF, 0.1% | | | |
| | | butylhydroxytoluene, 63.7980% | | | |
| | | Duro-Tak 387-2287 | | | |
| 17 | 17 | 11% risperidone, 5% K90, 10% oleic | 15 | 0 | 2 |
| | | acid, 1.0% EPO, 0.1% DL-alpha | | | |
| | | tocopherol, 0.002% ascorbyl | | | |
| | | palmitate NF, 0.1% | | | |
| | | butylhydroxytoluene, 72.7980% | | | |
| | | Duro-Tak 387-2287 | | | |
| 18 | 18 | 11% risperidone, 5% K90, 10% oleic | 14 | 0 | 2 |
| | | acid, 1.5% EPO, 0.1% DL-alpha | | | |
| | | tocopherol, 0.002% ascorbyl | | | |
| | | palmitate NF, 0.1% | | | |
| | | butylhydroxytoluene, 72.2980% | | | |
| | | Duro-Tak 387- 2287 | | | |
| 19 | 19 | 11% risperidone, 10% K90, 10% oleic | 9 | 0 | 1.5 |
| | | acid, 10% crospovidone, 0.5% EPO, | | | |
| | | 0.1% DL-alpha tocopherol, 0.002% | | | |
| | | ascorbyl palmitate NF, 0.1% | | | |
| | | butylhydroxytoluene, 58.2980% | | | |
| | | Duro-Tak 387-2287 | | | |
| 20 | 20 | 11% risperidone, 10% K90, 10% oleic | 9 | 0 | 2 |
| | | acid, 10% crospovidone, 0.75% EPO, | | | |
| | | 0.1% DL-alpha tocopherol, 0.002% | | | |
| | | ascorbyl palmitate NF, 0.1% | | | |
| | | butylhydroxytoluene, 58.0480% | | | |
| | | Duro-Tak 387-2287 | | | |
| 21 | 21 | 11% risperidone, 10% oleic acid, | 15 | 20% | Not carried |
| | | 2.25% EPO, 0.1% DL-alpha | | | out |
| | | tocopherol, 0.1% | | | |
| | | butylhydroxytoluene, 76.30% Duro- | | | |
| | | Tak 387-2287 | | | |
| 22 | 22 | 11% risperidone, 10% oleic acid, | 15 | 0 | 2 |
| | | 2.75% EPO, 0.1% DL-alpha | | | |
| | | tocopherol, 0.1% | | | |
| | | butylhydroxytoluene, 75.80% Duro- | | | |
| | | Tak 387-2287 | | | |
| 23 | 23 | 11% risperidone, 10% oleic acid, 1.5% | 15 | 0 | 2 |
| | | EPO, 0.1% DL-alpha tocopherol, 0.1% | | | |
| | | butylhydroxytoluene, 77.3% Duro- | | | |
| | | Tak 387-2287 | | | |

### Patch Wearing Test

One healthy volunteer participated in the patch wearing study. The skin of the left and right thighs was cleaned with wet paper towels and dried with dry paper towels. In each study, one or more patches were applied to the left and right thighs. After applying the patch, it was pressed with the palm for 30 seconds. The start date and time of the experiment were recorded. The adhesion score was recorded. The results are shown in Table 7, and the adhesion was scored using a five-point scale from 0 to 4.

Skin adhesion scores from the patch study are reported in Table 6. Most preparations have an adhesion score of 2 with no black rings caused by cold flow. The data show that the black rings caused by cold flow can be zeroed out by adjusting the amount of cohesion promoter. But its adhesion cannot meet the requirements.

TABLE 7
Skin stickiness score

| Degree of adhesion | Fraction |
|---|---|
| ≥90% (basically no lifting from the skin) | 0 |
| ≥75% to <90% (only some edges lifting up from skin) | 1 |
| ≥50% to <75% (less than half of the patch | 2 |
| lifting from the skin) | |
| >0% to <50% (more than half of the patch | 3 |
| lifting the skin but not detaching) | |
| 0% (the patch completely separates from the skin) | 4 |

### Examples 24 and 25

Each component was weighed according to Table 8. K 90, isostearic acid or stearic acid, EPO, DL-alpha tocopherol, and 0.1% butylated hydroxytoluene were dissolved in isopropanol. The solution was mixed for 24 hours. Duro-Tak 387-2287 was added and mixed well. Micronized risperidone crystal form A was added to form a homogeneous suspension in which undissolved risperidone crystals are suspended. The wet blend was coated on a release film, dried, and then laminated to a backing film Scotchpak 9738.

TABLE 8

| Example# | Preparation# | Preparation composition |
|---|---|---|
| 24 | 24 | 11% risperidone, 10% isostearic acid, |
| | | 1.5% EPO, 0.1% DL-alpha |
| | | tocopherol, 0.1% butylhydroxytoluene, |
| | | 77.30% Duro-Tak 387-2287 |
| 25 | 25 | 11% risperidone, 10% stearic acid, |
| | | 1.5% EPO, 0.1% DL-alpha |
| | | tocopherol, 0.1% butylhydroxytoluene, |
| | | 77.30% Duro-Tak 387-2287 |

### Example 26 Matrix-Type Transdermal Administration System Using Overlapping Adhesive Films

Duro-Tak 387-2516 (10% w/w) and Duro-Tak 387-2287 (90% w/w) were mixed evenly, and then the mixture was coated on the release film, and the coated release film was dried (40° C. 4 min, 85° C. 4 min) and then compounded on the separation layer polyester fabric KOB053, with a coating thickness of 110 gsm, to prepare a backing layer/overlapping adhesive film/release film composite. The release film layer of the backing layer/overlapping adhesive film/release film composite was removed and the separation layer of the 10 cm² drug patch from example 23 was applied to the overlapping adhesive film. The release film was removed from the 10 cm² drug adhesive layer (drug matrix layer) and an oversized release film was applied on the drug adhesive layer and overlapping adhesive film layer, the final patch was die-cut so that the overlapping adhesive film layer extended 10 mm beyond the separation layer and the drug adhesive layer in each direction.

### Comparative Example 7

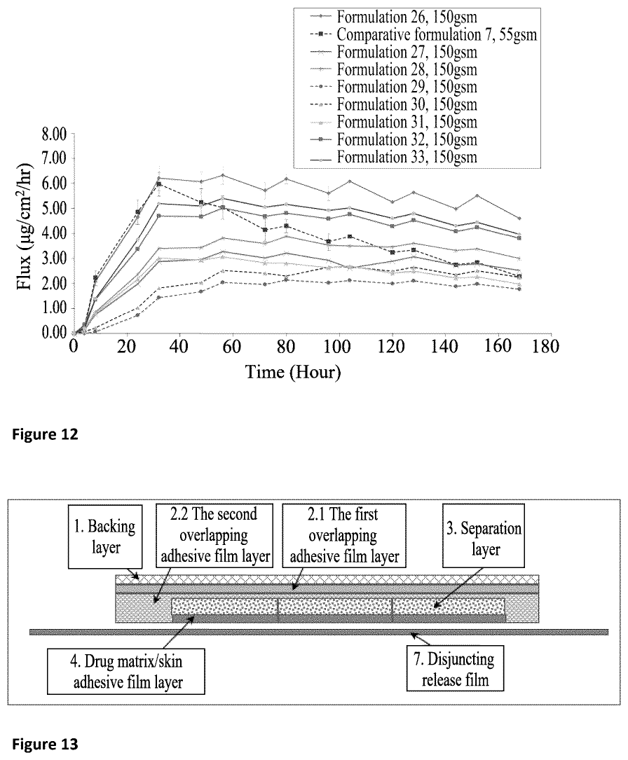

Comparative formulation 7 patches were prepared according to example 4 of patent CN 101366705B. As shown in Table 9 and FIG. 12, the skin flux of comparative formulation 7 decreased rapidly after 48 hours, and the skin flux from 24 to 168 hours was not constant, but continuously decreasing. The skin flux at 168 hours was 61% lower than the maximum skin flux.

### Examples 26 to 33

Lauryl lactate, oleyl alcohol, EPO, DL-alpha tocopherol, 0.002% ascorbyl palmitate NF, 0.1% butylated hydroxytoluene were dissolved in isopropanol. The solution was mixed for 24 hours. Duro-Tak 387-2287 was added and mixed well. Micronized risperidone crystal form A was added to form a homogeneous suspension in which undissolved risperidone crystals are suspended. The wet blend was coated on a release film, dried, and then laminated to a backing layer film Scotchpak 1109. 5 laminated materials were prepared with a coating weight of 150 gsm. Microscopic analysis showed that the laminated plate was filled with crystals. As shown in Table 9 and FIG. 12, the skin flux from 24 hours to 168 hours was not only basically constant, but the transdermal amount at 168 hours was maintained at more than 70% of the maximum transdermal amount of risperidone.

TABLE 9
Skin flux of examples 26 to 33 and comparative example 7
Average value of skin from three donors

| | Example 26 | | | | |
|---|---|---|---|---|---|
| | Formulation 26 | Comparative | Example 27 | Example 28 | Example 29 |
| | 11% risperidone, | example 7 | Formulation 27 | Formulation 28 | Formulation 29 |
| | 20% lauryl | Comparative | 11% risperidone, | 11% risperidone, | 11% risperidone, |
| | lactate, | formulation 7 | 12.5% lauryl | 15% lauryl | 10% oleyl |
| | 2.25% EPO, | 9% risperidone, | lactate, 2% | lactate, 2% | alcohol, 1.25% |
| | 0.1% DL- | 6% azone, 8% | EPO, 0.1% | EPO, 0.1% | EPO, 0.1% |
| | alpha | propylene | DL-alpha | DL-alpha | DL-alpha |
| | tocopherol, | glycol, 5% | tocopherol, | tocopherol, | tocopherol, |
| | 0.1% BHT, | 1-dodecanol, | 0.1% BHT, | 0.1% BHT, | 0.1% BHT, |
| | 66.5500% | 72.0000% | 74.3000% | 71.8000% | 77.5500% |
| time | Duro-Tak 387- | Duro-Tak 387- | Duro-Tak 387- | Duro-Tak387- | Duro-Tak 387- |
| (hours) | 2287, 150 gsm | 2287, 55 gsm | 2287, 150 gsm | 2287, 150 gsm | 2287, 150 gsm |
| 0 | 0.000 | 0.000 | 0.000 | 0.000 | 0.000 |
| 4 | 0.262 | 0.309 | 0.144 | 0.171 | 0.000 |
| 8 | 2.076 | 2.222 | 0.746 | 0.858 | 0.071 |
| 24 | 4.642 | 4.848 | 1.983 | 2.359 | 0.726 |
| 32 | 6.206 | 5.970 | 2.864 | 3.398 | 1.442 |
| 48 | 6.054 | 5.237 | 2.939 | 3.419 | 1.670 |
| 56 | 6.320 | 5.036 | 3.271 | 3.804 | 2.035 |
| 72 | 5.718 | 4.133 | 3.013 | 3.582 | 1.961 |
| 80 | 6.181 | 4.301 | 3.206 | 3.867 | 2.139 |
| 96 | 5.600 | 3.654 | 2.915 | 3.512 | 2.019 |
| 104 | 6.083 | 3.875 | 2.608 | 3.479 | 2.112 |
| 120 | 5.245 | 3.232 | 2.884 | 3.450 | 1.988 |
| 128 | 5.626 | 3.326 | 3.061 | 3.595 | 2.096 |
| 144 | 4.975 | 2.737 | 2.720 | 3.306 | 1.874 |
| 152 | 5.504 | 2.833 | 2.767 | 3.370 | 1.975 |
| 168 | 4.602 | 2.277 | 2.523 | 2.996 | 1.769 |
| Decline % | 27.18% | 61.86% | 22.87% | 22.51% | 17.32% |

Risperidone = risperidone,
Eudragit EPO,
DL-alpha tocopherol = DL-α tocopherol,
Lauryl lactate = lauryl lactate,
oleyl alcohol = oleyl alcohol,
Propylene glycol = propylene glycol,
Azone = azone
1-Dodecanol = 1-dodecanol,
gsm = grams per square meter
Decline % = (Cmax − C168 h)/Cmax*100

TABLE 9-2

| | Example 30 | Example 31 | Example 32 | Example 33 |
|---|---|---|---|---|
| | Formulation 30 | Formulation 31 | Formulation 32 | Formulation 33 |
| | 11% risperidone, | 11% risperidone, | 11% risperidone, 5% | 11% risperidone, |
| | 10% lauryl lactate, | 5% lauryl lactate, | lauryl lactate, 10.8% | 10% lauryl lactate, |
| | 1.25% EPO, | 1.5% EPO, | oleyl alcohol, 2.5% | 10.8% oleyl alcohol, |
| | 0.1% DL-alpha | 0.1% DL-alpha | EPO, 0.1% DL-alpha | 2.75% EPO, 0.1% DL- |
| | tocopherol, 0.1% | tocopherol, 0.1% | tocopherol 1, 0.1% | alpha tocopherol, |
| | BHT, 77.5500% | BHT, 82.3000% | BHT, 70.5000% | 0.1% BHT, 65.2500% |
| time | Duro-Tak 387 | Duro-Tak 387- | Duro-Tak 387-2287, | Duro-Tak 387-2287, |
| (hours) | 2287, 150 gsm | 2287, 150 gsm | 150 gsm | 150 gsm |
| 0 | 0.000 | 0.000 | 0.000 | 0.000 |
| 4 | 0.072 | 0.187 | 0.360 | 0.191 |
| 8 | 0.234 | 0.821 | 1.360 | 1.380 |
| 24 | 1.037 | 2.163 | 3.358 | 3.739 |
| 32 | 1.823 | 3.005 | 4.687 | 5.184 |
| 48 | 2.045 | 2.922 | 4.656 | 5.090 |
| 56 | 2.520 | 3.052 | 5.014 | 5.395 |
| 72 | 2.403 | 2.824 | 4.674 | 5.051 |
| 80 | 2.295 | 2.795 | 4.806 | 5.155 |
| 96 | 2.650 | 2.622 | 4.583 | 4.918 |
| 104 | 2.654 | 2.687 | 4.751 | 5.007 |
| 120 | 2.488 | 2.398 | 4.284 | 4.591 |
| 128 | 2.644 | 2.483 | 4.513 | 4.793 |
| 144 | 2.347 | 2.214 | 4.071 | 4.301 |
| 152 | 2.497 | 2.260 | 4.233 | 4.438 |
| 168 | 2.242 | 1.980 | 3.781 | 3.956 |
| Decline % | 15.52% | 35.12% | 24.59% | 26.68% |

### Example 34 Matrix-Type Transdermal Administration System Using Overlapping Adhesive Films

Duro-Tak 387-2051 and Duro-Tak 387-2054 were mixed evenly, and then the mixture was coated on the release film, and the coated release film was dried (40° C. 4 min, 85° C. 4 min) and then compounded on the backing layer KOB053 to prepare a backing layer/overlapping adhesive film/release film composite. The release film layer of the backing layer/overlapping adhesive film/release film composite was removed and the separation layer of the 10 cm² drug patch from example 23 was applied to the overlapping adhesive film. The release film was removed from the 10 cm² drug adhesive layer (drug matrix layer) and an oversized release film was applied on the drug adhesive layer and overlapping adhesive film layer, the final patch was die-cut so that the overlapping adhesive film layer extended 10 mm beyond the separation layer and the drug adhesive layer in each direction.

### Example 35 Matrix-Type Transdermal Administration System Using Overlapping Adhesive Films

The matrix-type transdermal administration system of example 35 was prepared using a method similar to that of example 34. The backing layer KOB053/(Duro-Tak 387-2054+Duro-Tak 387-2051) in example 34 was replaced with the backing layer KOB 053/(Duro-Tak 387-2516+Duro-Tak 387-2287).

### Example 36 Matrix-Type Transdermal Administration System Using Overlapping Adhesive Films

Duro-Tak 387-2516 (10% w/w) and Duro-Tak 387-2287 (90% w/w) were mixed evenly, and then the mixture was coated on the release film, and the coated release film was dried (40° C. 4 min, 85° C. 4 min) and then compounded on the backing layer KOB053, and the DURO-TAK 387-2516 and DURO-TAK 387-2287 mixed adhesive layer was covered with a polyisobutylene adhesive layer. A backing layer/overlapping adhesive film/release film composite was prepared. The release film layer of the backing layer/overlapping adhesive film/release film composite was removed and the separation layer of the 10 cm² drug patch from example 23 was applied to the overlapping adhesive film. The release film was removed from the 10 cm² drug adhesive layer (drug matrix layer) and an oversized release film was applied on the drug adhesive layer and overlapping adhesive film layer, the final patch was die-cut so that the overlapping adhesive film layer extended 10 mm beyond the separation layer and the drug adhesive layer in each direction.

### Example 37 Matrix-Type Transdermal Administration System Using Overlapping Adhesive Films

The matrix-type transdermal administration system of example 37 was prepared using a method similar to that of example 36. The backing layer KOB (/Duro-Tak 387-2516+Duro-Tak 387-2287)/PIB in example 36 was replaced with the backing layer KOB053/(Duro-Tak 387-2516+Duro-Tak 387-2287)/Bio-PSA 7-4301.

### Example 38 Matrix-Type Transdermal Administration System Using Overlapping Adhesive Films

The matrix-type transdermal administration system of example 38 was prepared using a method similar to that of example 36. The backing layer KOB (/Duro-Tak 387-2516+Duro-Tak 387-2287)/PIB in example 36 was replaced with the backing layer KOB053/(387-2516+387-2287)/SIS.

The test results of the migration of the non-volatile component risperidone from the central drug-containing layer to the overlapping adhesive film layer in examples 34-38 are summarized in Table 10.

Example 34: As shown in Table 10, when the adhesive of the overlapping adhesive film layer is a blend of two acrylic ester adhesives (Duro-Tak 387-2054, Duro-Tak 387-2051), a considerable amount of non-volatile risperidone migrates from the central drug-containing layer to the overlapping adhesive film layer.

Example 35: As shown in Table 10, when the adhesive of the overlapping adhesive film layer is a blend of two other acrylic ester adhesives (Duro-Tak 387-2516, Duro-Tak 387-2287), a considerable amount of non-volatile risperidone migrates from the central drug-containing layer to the overlapping adhesive film layer.

Example 36, example 37 and example 38 It was unexpectedly found that when the overlapping adhesive film layer in contact with the central backing layer was the siloxane Bio-PSA 7-4301, styrene-isoprene-styrene or polyisobutylene, there was little non-volatile risperidone migrates from the central drug-containing layer to the overlapping adhesive film layer.

TABLE 10
Migration of the non-volatile component risperidone from the central
drug-containing layer to the overlapping adhesive film layer

| | Risperidone content of | | | |
|---|---|---|---|---|
| | covering adhesive layer | | | |
| | 60° C. | 60° C. | 60° C. | 40° C. |
| | for 3 | for 5 | for 10 | for one |
| Time point | days | days | days | month |
| Example 34, overlapping adhesive film layer: backing | 0.60% | 0.80% | 1.20% | 0.70% |
| layer KOB053/(Duro-Tak 387-2054 + Duro-Tak 387-2051) | | | | |
| Example 35, overlapping adhesive film layer: backing | 0.90% | 1.30% | 1.40% | 0.90% |
| layer KOB 053/(Duro-Tak 387-2516 + Duro-Tak 387-2287) | | | | |
| Example 36, overlapping adhesive film layer: backing | 0.07% | 0.09% | 0.10% | 0.10% |
| layer KOB(/Duro-Tak 387-2516 + Duro-Tak 387-2287)/PIB | | | | |
| Example 37, overlapping adhesive film layer: backing | 0.06% | 0.09% | 0.11% | 0.09% |
| layer KOB053/(Duro-Tak 387-2516 + Duro-Tak 387- | | | | |
| 2287)/Bio-PSA 7-4301 | | | | |
| Example 38, overlapping adhesive film layer: backing | 0.07% | 0.08% | 0.10% | 0.10% |
| layer KOB053/(387-2516 + 387-2287)/SIS | | | | |

PIB = polyisobutylene;
SIS = styrene-isoprene-styrene copolymer

### Comparative Example 8 the Separation Layer and the Drug Matrix Layer Did not have a Plurality of Small Pieces

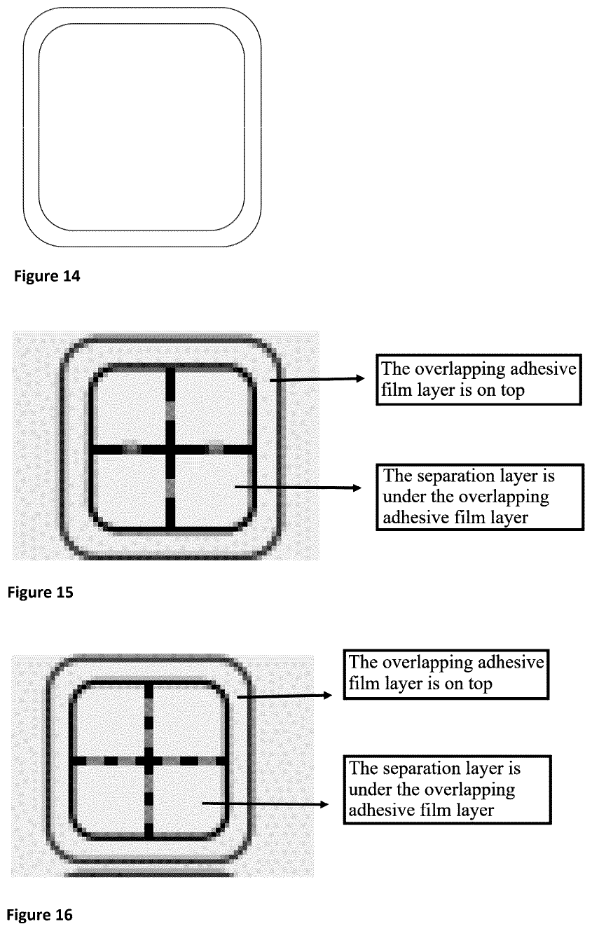

As shown in FIG. 14, a transdermal patch includes: (a) a backing layer of 82 cm² composed of an elastic material; (b) an overlapping adhesive layer of 82 cm²; (c) a separation layer of 50 cm²; (d) a drug adhesion layer of 50 cm²; (e) a release film. The separation layer and the drug matrix layer were not semi-cut into a plurality of small pieces. 15 volunteers completed 7 days of wear, and at the day 7 time point, the adhesion score for the edge of the overlapping layer was 0.1, but the adhesion score for the inner patch was 1.

### Example 39 A Plurality of Small Pieces of the Separation Layer and the Drug Matrix Layer had One Connection Point

Example 39 is as shown in FIG. 15, a transdermal patch includes: (a) a backing layer of 82 cm² composed of an elastic material; (b) an overlapping adhesive layer of 82 cm²; (c) a separation layer of 50 cm²; (d) a drug adhesion layer of 50 cm²; (e) a release film. The separation layer and drug matrix layer were semi-cut into 4 small pieces. Among them, each small piece of the separation layer had one connection point. At the day 7 wear time point, the adhesion score for the edge of the overlapping layer was 0.1, but the adhesion score for the inner patch was 0.3.

### Example 40 A Plurality of Small Pieces of the Separation Layer and the Drug Matrix Layer had Two Connection Points

As shown in FIG. 16, a transdermal patch includes: (a) a backing layer of 82 cm² composed of an elastic material; (b) an overlapping adhesive layer of 82 cm²; (c) a separation layer of 50 cm²; (d) a drug adhesion layer of 50 cm²; (e) a release film. The separation layer and drug matrix layer were semi-cut into 4 small pieces. Among them, each small piece of the separation layer had two connection points. At the day 7 wear time point, the adhesion score for the edge of the overlapping layer was 0.1, but the adhesion score for the inner patch was 0.3.

### Comparative Example 9 Small Pieces without Connection Points were Arranged Out of Order During Transfer

As shown in FIG. 18, the separation layer and drug matrix layer are semi-cut into 4 pieces of 12.5 cm² on a die-cutting machine. There are no connection points between small pieces (grey); The intersecting black lines are the cut separation layer and drug matrix layer. When the inner patch (separation layer and matrix layer) moves forward, the 4 parts of the inner patch are separated from each other and arranged in disorder.

### Example 41 Small Pieces with Two Connection Points Achieved Orderly Arrangement During Transfer

As shown in FIG. 19, the separation layer and drug matrix layer are cut into 4 small pieces of 12.5 cm² on a die-cutting machine. there are connection points between small pieces (grey); the intersecting black lines are the cut separation layer and drug matrix layer. When the inner patch (separation layer and matrix layer) moves forward, the 4 small parts of the inner patch are connected together in order.

### Example 42 to Example 45 Skin Flux Test of Patch

The transdermal patch of Examples 42-45 has the structure shown in FIG. 4B, in which the drug matrix was prepared using formulation 42 to formulation 45 in Table 11, risperidone was micronized risperidone crystal form A, and the first overlapping adhesive film was acrylic adhesive Duro-Tak 87-2054 at 55 GSM, the second overlapping adhesive film was polyisobutylene at 55 GSM, the separation layer was Scotchpak 1109, and the backing layer was KOB053. The matrix layer and separation layer after film-cut had connection points as shown in FIGS. 16 and 19.

TABLE 11
Skin flux of example 42 to example 45

| | Average value of skin from three donors | | | |
|---|---|---|---|---|
| | Example 42 | Example 43 | Example 44 | Example 45 |
| | Formulation 42 | Formulation 43 | Formulation 44 | Formulation 45 |
| | 11% risperidone, | 11% risperidone, | 11% risperidone, | 11% risperidone, |
| | 4.12% lauryl lactate, | 4.12% lauryl lactate, | 4.12% lauryl lactate, | 4.12% lauryl lactate, |
| | 10% oleyl alcohol, | 10% oleyl alcohol, | 10% oleyl alcohol, | 10% oleyl alcohol, |
| | 2.5% EPO, 0.1% DL- | 2.5% EPO, 0.1% DL- | 2.5% EPO, 0.1% DL- | 2.5% EPO, 0.1% DL- |
| | alpha tocopherol, | alpha tocopherol, | alpha tocopherol, | alpha tocopherol, |
| | 0.1% BHT, 72.18% | 0.1% BHT, 72.18% | 0.1% BHT, 72.18% | 0.1% BHT, 72.18% |
| time | Duro-Tak 387-2287, | Duro-Tak 387-2287, | Duro-Tak 387-2287, | Duro-Tak 387-2287, |
| (hours) | 50 gsm | 75 gsm | 100 gsm | 150 gsm |
| 0 | 0.000 | 0.000 | 0.000 | 0.000 |
| 4 | 0.000 | 0.012 | 0.010 | 0.033 |
| 8 | 0.162 | 0.200 | 0.266 | 0.303 |
| 24 | 1.296 | 1.508 | 1.598 | 1.490 |
| 32 | 2.406 | 2.685 | 2.718 | 2.641 |
| 48 | 2.605 | 2.910 | 2.949 | 2.685 |
| 56 | 2.756 | 3.047 | 3.098 | 2.773 |
| 72 | 2.514 | 2.872 | 2.858 | 2.622 |
| 80 | 2.657 | 2.880 | 2.881 | 2.659 |
| 96 | 2.599 | 2.758 | 2.674 | 2.466 |
| 104 | 2.678 | 2.826 | 2.807 | 2.565 |
| 120 | 2.507 | 2.711 | 2.635 | 2.453 |
| 128 | 2.522 | 2.733 | 2.582 | 2.448 |
| 144 | 2.369 | 2.603 | 2.417 | 2.316 |
| 152 | 2.412 | 2.611 | 2.443 | 2.330 |
| 168 | 2.226 | 2.489 | 2.262 | 2.215 |
| Average flux | 2.114 | 2.323 | 2.280 | 2.133 |
| Decline % | 23.290 | 18.330 | 26.990 | 20.130 |

Risperidone = risperidone,
Povidone K90 = povidone K90,
Oleic acid = oleic acid,
Eudragit EPO,
DL-alpha tocopherol = DL-α tocopherol,
Ascorbyl palmitate NF = ascorbyl palmitate NF,
BHT = butylated hydroxytoluene,
Crospovidone CL-M = crospovidone CL-M,
Duro-Tak 387-2516 gsm = grams per square meter
Decline % = (Cmax − C168 h)/Cmax*100

1. A risperidone transdermal administration system, comprising:
1) a backing layer;
2) a matrix layer, which contains risperidone dispersed in the matrix layer in crystalline and non-crystalline, a skin penetration enhancer and a pressure-sensitive adhesive; and
3) a release liner layer.

2. The risperidone transdermal administration system of claim 1, wherein the crystalline form of the risperidone is crystalline form A, preferably micronized risperidone crystalline form A.

3. The risperidone transdermal administration system of claim 1 or 2, wherein the skin penetration enhancer is selected from one or more of C₁-C₃₀ fatty acids, fatty esters, or fatty alcohols;
preferably, the fatty acids are selected from C₁₂-C₂₂ unsaturated fatty acids, more preferably oleic acid;
preferably, the fatty esters are selected from esters produced by C₁-C₃₀ fatty acids and C₁-C₃₀ alcohols in combination, more preferably lauryl lactate,
preferably, the fatty alcohols are selected from C₁₃-C₃₀ alkenyl alcohols, more preferably oleyl alcohol.

4. The risperidone transdermal administration system of any one of claims 1-3, wherein the matrix layer includes the following components relative to the total weight of the matrix layer:
1) risperidone at a total amount of 9.5-15%, preferably 9.5-15% or 10-12%;
2) the skin penetration enhancer at an amount of 1%-50%, preferably 2-30%, 8.5-30%, 8.5-25%, or 8.5-15%; more preferably, the C₁-C₃₀ fatty acids at an amount of 8.5-15%, the fatty esters at an amount of 1-10%, and the fatty alcohols at an amount of 1-15%; and most preferably, the oleic acid at an amount of 8.5-15%, the lauryl lactate at an amount of 1-10%, 1-6% or 3-5%, and the C₁₃-C₃₀ alkenyl alcohols at an amount of 1-15%, 5-15% or 8-15%;
3) the pressure-sensitive adhesive at an amount of 50-82%, preferably 50-80% or 55-80%;
with the total amount of all component in the matrix layer being 100%.

5. The risperidone transdermal administration system of any one of claims 1-4, wherein the matrix layer consists of the following components relative to the total weight of the matrix layer:
1) risperidone at a total amount of 9.5-15%, preferably 9.5-15% or 10-12%;
2) the skin penetration enhancer at an amount of 1-50%, preferably 2-30%, 8.5-30%, 8.5-25%, 8.5-15%; more preferably, the C₁-C₃₀ fatty acids at an amount of 8.5-15%, the fatty esters at an amount of 1-10%, and the fatty alcohols at an amount of 1-15%; and most preferably, oleic acid at an amount of 8.5-15%, lauryl lactate at an amount of 1-10%, 1-6% or 3-5%, C₁₃-C₃₀ alkenyl alcohol at an amount of 1-15%, 5-15% or 8-15%;
3) the pressure-sensitive adhesive at an amount of 50-82%, preferably 50-80% or 55-80%;
4) an antioxidant at an amount of 0%-1%, preferably 0.05%-0.5% or 0.1%-0.3%;
5) a cohesion promoter at an amount of 0-30%, preferably 1-5%;
6) a tackifier at an amount of 0-40%;
7) a plasticizer at an amount of 0-40%;
8) an additional penetration enhancer at an amount of 0-10%;
the total amount of all component in the matrix layer is 100%.

6. The risperidone transdermal administration system of claim 5, wherein the pressure-sensitive adhesive is selected from one or more of acrylic adhesives, organosilicon adhesives, acrylic-organosilicon copolymer adhesives, polybutylene adhesives, styrene-isoprene-butylene copolymers, and styrene-butadiene-styrene copolymers.

7. The risperidone transdermal administration system of claim 5, wherein the cohesion promoter is selected from crospovidone, Eudragit E, Eudragit EPO, Eudragit S, Eudragit R. Plastoid B and mixtures thereof.

8. The risperidone transdermal administration system of claim 5, wherein the cohesion promotor is povidone K90, Eudragit EPO, and combinations of povidone K90 and Eudragit EPO, crospovidone CL-M and Eudragit EPO, povidone K90 and crospovidone CL-M and Eudragit EPO, povidone K90 and Plastoid B, crospovidone CL-M and Plastoid B, povidone K90 and crospovidone CL-M, and a preferred combination is povidone K90 and Eudragit EPO.

9. The risperidone transdermal administration system of any one of claims 5-8, wherein the antioxidant is selected from one or more of tocopherol, tocopheryl acetate, potassium metabisulfite, sodium metabisulfite, sodium bisulfite, sodium sulfite, propyl gallate, thioglycerol, sodium thiosulfate, sodium dioxide, sodium formaldehyde sulfoxylate, and butylated hydroxytoluene (BHT); and further preferably, a chelating agent as a synergistic antioxidant selecting from citric acid, tartaric acid, calcium disodium edetate, disodium edetate, and EDTA.

10. The risperidone transdermal administration system of any one of claims 5-9, wherein the tackifier is selected from polybutenes, terpenes, and mixtures thereof.

11. The risperidone transdermal administration system of any one of claims 5-10, wherein the plasticizer is selected from mineral oil, silicone oil, triethyl citrate, and mixtures thereof.

12. The risperidone transdermal administration system of any one of claims 1-11, wherein the amount of the matrix layer is from 30 g/m² to 700 g/m².

13. The risperidone transdermal administration system of any one of claims 1-12, wherein during the preparation process, risperidone is added in the form of micronized risperidone crystal form A, and preferably the particle size of the micronized risperidone is less than or equal to 20 μm.

14. The risperidone transdermal administration system of any one of claims 1-13, wherein a solubilizer or a solvent is further used in the preparation of the matrix layer, and is selected from C₁-C₆ alkyl alcohol, n-heptane, ethyl acetate, toluene and mixtures thereof; the solubilizer or the solvent is preferably ethanol, isopropanol, n-heptane or ethyl acetate.

15. The risperidone transdermal administration system of any one of claims 1-14, comprising:
1) a backing layer;
2) a matrix layer, which contains risperidone dispersed in the matrix layer in crystalline and non-crystalline, a skin penetration enhancer and a pressure-sensitive adhesive;
3) a skin-contacting adhesive layer; and
4) a release liner layer.

16. The risperidone transdermal administration system of any one of claims 1-14, comprising:
1) a backing layer;
2) a matrix layer, which contains risperidone dispersed in the matrix layer in crystalline and non-crystalline, a skin penetration enhancer and a pressure-sensitive adhesive;
3) a semipermeable membrane or machine-woven fabric layer;
4) a skin-contacting adhesive layer; and
5) a release liner layer.

17. The risperidone transdermal administration system of claim 16 or 17, wherein the skin-contacting adhesive layer includes a pressure-sensitive adhesive and optionally risperidone and optionally other excipients selected from one or more of a cohesion promoter, an antioxidant, an anti-skin irritation agent, a tackifier, a plasticizer, a solubilizer, a solvent and a desiccant.

18. The risperidone transdermal administration system of any one of claims 1-14, comprising:
1) a backing layer;
2) an overlapping adhesive film layer, preferably the material of the overlapping adhesive film layer is selected from one or more of siloxane, polyisobutylene, or styrene-isoprene-styrene copolymer; preferably, the amount of the overlapping adhesive film layer is 50-110 GSM;
3) a separation layer;
4) a matrix layer, which contains risperidone dispersed in the matrix layer in crystalline and non-crystalline, a skin penetration enhancer and a pressure-sensitive adhesive; and
5) a release liner layer.

19. The risperidone transdermal administration system of claim 18, wherein the overlapping adhesive film layer includes an adhesive, and the adhesive of the overlapping adhesive film layer is the same as or different from the adhesive of the drug matrix layer; preferably, the solubility of a drug in the adhesive of the overlapping adhesive film layer is the same as or less than that of the drug in the adhesive of the matrix layer; more preferably, the solubility of the drug in the adhesive of the overlapping adhesive film layer is less than that of the drug in the adhesive of the matrix layer.

20. The risperidone transdermal administration system of claim 18 or 19, wherein the overlapping adhesive film layer can be one layer, two layers or multiple layers; preferably, the first overlapping adhesive film layer close to the backing layer can prevent other overlapping adhesive film layers away from the backing layer from migrating to the backing layer or the side of the backing layer away from the skin.

21. The risperidone transdermal administration system of claim 20, wherein the adhesive of the first overlapping adhesive film layer is selected from Duro-Tak 387-2516, Duro-Tak 387-2287, Duro-Tak 387-4287, Duro-Tak 87-2051, Duro-Tak 87-2052, Duro-Tak 87-2054, Duro-Tak 87-2852 or mixtures thereof; the adhesive of the second overlapping adhesive film layer is selected from polyisobutylene, styrene-isoprene-styrene copolymer, dimethylsiloxane, or mixtures thereof.

22. The risperidone transdermal administration system of any one of claims 18-21, wherein the risperidone transdermal administration system, from a backing layer to a disjuncting layer, includes: a backing layer, one or more overlapping adhesive film layers, a separation layer, a matrix layer, and a release liner layer; the backing layer, the one or more overlapping adhesive film layers and the release liner layer extend all around beyond the separation layer and the matrix layer; during application, the disjuncting layer is removed and the matrix layer in contact with the skin is sealed to the skin by one or more overlapping adhesive film layers.

23. The risperidone transdermal administration system of claim 22, wherein the overlapping adhesive film layer is one layer, and the risperidone transdermal administration system, from a backing layer to a disjuncting layer, includes:
i. a backing layer, an overlapping adhesive film layer, a separation layer, a matrix layer, and a release liner layer, and the backing layer, the overlapping adhesive film layer, and the release liner layer extend all around beyond the separation layer and the matrix layer; the structure illustrated in FIG. 4A is preferred; or
ii. a backing layer, an overlapping adhesive film layer, a separation layer, a matrix layer, a skin adhesive layer, and a release liner layer, and the backing layer, the overlapping adhesive film layer and the release liner layer extend all around beyond the separation layer, the matrix layer and the skin adhesive layer; the structure illustrated in FIG. 4C is preferred; or
iii. a backing layer, an overlapping adhesive film layer, a separation layer, a matrix layer, a semipermeable membrane layer, a skin adhesive layer, and a release liner layer; and the backing layer, the overlapping adhesive film layer and the release liner layer extend all around beyond the separation layer, the matrix layer, the semipermeable membrane layer and skin adhesive layer; the structure illustrated in FIG. 4F is preferred.

24. The risperidone transdermal administration system of claim 22, wherein the overlapping adhesive film layer is two layers; during application, the disjuncting layer is removed and the matrix layer in contact with the skin is sealed to the skin by the second overlapping adhesive film layer; the risperidone transdermal administration system, from a backing layer to a disjuncting layer, includes:
i. a backing layer, a first overlapping adhesive film layer, a second overlapping adhesive film layer, a separation layer, a matrix layer, and a release liner layer, and the backing layer, the first overlapping adhesive film layer, the second overlapping adhesive film layer and the release liner layer extend all around beyond the separation layer and the matrix layer; the structure illustrated in FIG. 4B is preferred; or
ii. a backing layer, a first overlapping adhesive film layer, a separation layer, a matrix layer, a skin adhesive layer, a release liner layer, and a backing layer, and the first overlapping adhesive film layer and the release liner layer extend all around beyond the separation layer, the matrix layer and the skin adhesive layer, the second overlapping adhesive film layer is filled between the first overlapping adhesive film layer and the release liner layer, the structure illustrated in FIG. 4D is preferred; or
iii. a backing layer, a first overlapping adhesive film layer, a separation layer, a matrix layer, a semipermeable membrane layer, a skin adhesive layer, a second overlapping adhesive film layer, and a release liner layer, and the backing layer, the first overlapping adhesive film layer, the second overlapping adhesive film layer and the release liner layer extend all around beyond the separation layer, the matrix layer, the semipermeable membrane layer and the skin adhesive layer, and the second overlapping adhesive film layer extends toward the first overlapping adhesive film layer and wraps the separation layer, the matrix layer, the semipermeable membrane layer and the skin adhesive layer; the structure illustrated in FIG. 4E is preferred;
preferably, the amount of the first overlapping adhesive film layer is 25-60 GSM; the amount of the second overlapping adhesive film layer is 25-60 GSM.

25. The risperidone transdermal administration system of any one of claims 18-24, wherein the backing layer is composed of a polymer elastic film, a polymer fabric, a bidirectional or multi-directional elastic nonwoven fabric, a stretchable polymer film, a stretchable machine-woven fabric, or a stretchable nonwoven fabric.

26. The risperidone transdermal administration system of claim 25, wherein the backing layer is selected from polyester, polyethylene, polypropylene, polyvinyl chloride, polyethylene vinyl acetate, or polyurethane, preferably KOB051 and KOB053.

27. The risperidone transdermal administration system of any one of claims 18-26, wherein the separation layer is selected from a flexible occlusive single-layer or multi-layer polymeric film, the polymer is selected from polyolefins, polyesters, polyethylene, polyvinylidene chloride or polyurethane; preferably, the separation layer further includes an aluminum film.

28. The risperidone transdermal administration system of any one of claims 18-27, wherein the separation layer includes a plurality of small pieces that are partially connected to each other, and the matrix layer includes a plurality of small pieces that are fully or partially connected;
preferably, the plurality of separation layer small pieces connected to each other in the separation layer are symmetrical or asymmetrical, the same or different, the plurality of small pieces connected to each other in the matrix layer are symmetrical or asymmetrical, the same or different; further preferably, a part of the connection points of the separation layer and a part of the connection points of the matrix layer are all aligned or partially aligned.

29. The risperidone transdermal administration system of claim 28, wherein there are one or more connection points between the adjacent small pieces, preferably 1, 2, 3, 4 or 5 connection points; and the length of each connection point is independently selected from the range of 0.5-3 mm.

30. The risperidone transdermal administration system of any one of claims 28-29, wherein overlapping separation layer pieces and matrix layer pieces have the same shape, and the number of the pieces in each transdermal administration system unit is selected from 1 to 10, each small piece has an area of 1-25 cm², preferably 1 cm², 4 cm², 6 cm², 8 cm², 9 cm², 10 cm², 12.5 cm², 16 cm², or 25 cm².

31. The risperidone transdermal administration system of any one of claims 1-30, wherein the matrix layer has a weight of 30 GSM to 700 GSM, 50 GSM to 700 GSM, 30 GSM to 100 GSM, 40 GSM to 150 GSM, 75 GSM to 150 GSM, 150 GSM to 300 GSM, or 350 GSM to 700 GSM.

32. A method for preparing the stable matrix-type risperidone transdermal administration system of any one of claims 1-14, comprising the following steps:
step 1. mixing a skin penetration enhancer and optionally a cohesion promoter, an antioxidant, an anti-skin irritation agent, a tackifier, a plasticizer, a solubilizer, a solvent and a desiccant to obtain blend 1;
step 2. mixing the blend 1 and a pressure-sensitive adhesive to obtain blend 2;
step 3. adding micronized risperidone crystal form A or a pharmaceutically acceptable salt thereof to the blend 2 from step 2, and stirring the resulting mixture until risperidone or the pharmaceutically acceptable salt thereof is evenly suspended to obtain a wet drug mixture;
step 4. coating the wet drug mixture on a release liner layer;
step 5. drying to remove the solvent and the solubilizer to obtain a release liner layer/drug matrix layer laminated material; and
step 6. laminating the release liner layer/drug matrix layer laminated material to the backing layer to form a release liner layer/drug matrix layer/backing layer composite film.

33. The method of claim 32, further comprising the following steps:
step 1. preparing a release liner layer/matrix layer/backing layer composite film according to steps 1 to 6 of the method of claim 32;
step 2. preparing a solution or a suspension of a skin-contacting adhesive layer containing a pressure-sensitive adhesive and optionally risperidone crystal form A or a pharmaceutically acceptable salt thereof and optionally other excipients, coating same to a release liner layer and drying to form a release liner layer/skin-contacting adhesive layer laminated material; and
step 3. removing the release liner layer from the release liner layer/matrix layer/backing layer composite film, and laminating the adhesive layer side of the release liner layer/skin-contacting adhesive layer of step 1 to the matrix layer to form a release liner layer/skin-contacting adhesive layer/matrix layer/backing layer composite film.

34. The method of claim 32, further comprising the following steps:
step 1. preparing a release liner layer/matrix layer/backing layer composite film according to steps 1 to 6 of the method of claim 32;
step 2. preparing a solution or a suspension of a skin-contacting adhesive layer containing a pressure-sensitive adhesive and optionally risperidone crystal form A or a pharmaceutically acceptable salt thereof and optionally other excipients, coating same to a release liner layer and drying to form a release liner layer/skin-contacting adhesive layer laminated material, and laminating the adhesive layer side to a semipermeable membrane or machine-woven fabric layer; and
step 3. removing the release liner layer from the release liner layer/matrix layer/backing layer composite film, and laminating the semipermeable membrane or machine-woven fabric layer side to the matrix layer to form a release liner layer/skin-contacting adhesive layer/semipermeable membrane or machine-woven fabric layer/matrix layer/backing layer composite film.

35. A method for preparing the risperidone transdermal administration system of any one of claim 18-23 or 25-31, comprising the following steps:
step A: coating the adhesive of the overlapping adhesive film layer on the release film, and after drying, compounding same to the backing layer to obtain a backing layer/overlapping adhesive film/release film composite;
step B: preparing the release liner layer/drug matrix layer laminated material according to steps 1 to 5 of claim 32, and laminating the release liner layer/drug matrix layer laminated material to the separation layer to form a release liner layer/drug matrix layer/separation layer film;
step C: removing the release film of the backing layer/overlapping adhesive film/release film composite of step A, and applying the separation layer of the release liner layer/drug matrix layer/separation layer film of step B to the overlapping adhesive film;
step D: removing the release film from the drug matrix layer and applying an oversized release film on the drug matrix layer and the overlapping adhesive film layer;
step E: die-cutting the final patch so that the overlapping adhesive film layer extends beyond the separation layer and the drug adhesive layer in each direction.

36. A method for preparing the risperidone transdermal administration system of claim 23, comprising the following steps
step A: coating the adhesive of the overlapping adhesive film layer on the release film, and after drying, compounding same to the backing layer to obtain a backing layer/overlapping adhesive film/release film composite;
step B: preparing a release liner layer/skin-contacting adhesive layer/drug matrix layer drug/separation layer composite film;
step C: removing the release film of the separation layer/overlapping adhesive film/release film composite of step A, and applying the separation layer of the release liner layer/skin-contacting adhesive layer/matrix layer/separation layer composite film formed in step B to the overlapping adhesive film;
step D: removing the release film from the skin-contacting adhesive layer and applying an oversized release film to the skin-contacting adhesive layer and the overlapping adhesive film layer;
step E: die-cutting the final patch so that the overlapping adhesive film layer extends beyond the separation layer, the matrix layer and the skin adhesive layer in each direction; preferably, the structure of FIG. 4C is formed.

37. A method for preparing the risperidone transdermal administration system of claim 23, comprising the following steps
step A: coating the adhesive of the overlapping adhesive film layer on the release film, and after drying, compounding same to the backing layer to obtain a backing layer/overlapping adhesive film/release film composite;
step B: preparing the release liner layer/skin-contacting adhesive layer/semipermeable membrane or machine-woven fabric layer/drug matrix layer/separation layer composite film;
step C: removing the release film of the backing layer/overlapping adhesive film/release film composite of step A, and applying the separation layer of the release liner layer/skin-contacting adhesive layer/semipermeable membrane or machine-woven fabric layer/drug matrix layer/separation layer composite film of step B to the overlapping adhesive film;
step D: removing the release film from the skin-contacting adhesive layer and applying an oversized release film to the skin-contacting adhesive layer and the overlapping adhesive film layer;
step E: die-cutting the final patch so that the backing layer, the overlapping adhesive film layer and the release liner layer extend all around beyond the separation layer, the matrix layer, the semipermeable membrane or machine-woven fabric layer and the skin adhesive layer;
preferably, the structure of FIG. 4F is formed.

38. A method for preparing the risperidone transdermal administration system of claim 24, comprising the following steps:
step A: coating the adhesive of the first overlapping adhesive film layer on the release film, and after drying, compounding same to the backing layer to obtain a backing layer/first overlapping adhesive film/release film composite film; coating the adhesive of the second overlapping adhesive film layer on the release film, and after drying, compounding same to the first overlapping adhesive film layer from which the release film is removed to obtain a backing layer/first overlapping adhesive film/second overlapping adhesive film/release film composite film;
step B: preparing a release liner layer/drug matrix layer/separation layer film, a release liner layer/skin adhesive layer/drug matrix layer/separation layer film, or a release liner layer/skin adhesive layer/semipermeable membrane machine-woven fabric layer/drug matrix layer/separation layer film;
step C: removing the release film of the backing layer/first overlapping adhesive film/second overlapping adhesive film/release film composite film of step A, and applying the separation layer of step B to the second overlapping adhesive film layer;
step D: removing the remaining release film of step C and applying an oversized release film on the side away from the backing layer;
step E: die-cutting the final patch so that the second overlapping adhesive film layer extends beyond the separation layer, the drug matrix layer, and optionally the skin adhesive layer and the semipermeable membrane machine-woven fabric layer in each direction; preferably, the structures of FIGS. 4B, 4D, and 4E are obtained.

39. A method for preparing the risperidone transdermal administration system of any one of claims 28-32, wherein
after die-cutting the composite film prepared in step B of claims 35-38 into a plurality of small pieces that are partially connected, and the structure of the small pieces of claims 28-31 is obtained; the structure in step C improves the arrangement order of the composite film in step B during transfer.

40. The method of any one of claims 32-39, wherein the wet drug mixture used to prepare the matrix layer contains undissolved risperidone crystal form A.

41. The method of any one of claims 32-40, wherein the solvent in step 1 or step B is selected from C₁-C₆ alkyl alcohol, n-heptane, ethyl acetate, toluene, and mixtures thereof; preferably, ethanol, isopropanol, n-heptane or ethyl acetate.

42. Use of a therapeutically effective amount of the risperidone transdermal administration system of any one of claims 1-41 in the preparation of a drug for treating or preventing schizophrenia, mania and dementia.

43. Use of a therapeutically effective amount of the risperidone transdermal administration system of any one of claims 1-41 in the preparation of a drug for treating or preventing positive and negative symptoms of schizophrenia.

44. The use according to claim 43, wherein the positive and negative symptoms of schizophrenia include hallucinations, delusions, and emotional withdrawal and blunted affect.

45. A method for treating or preventing schizophrenia, mania and dementia, comprising administering a therapeutically effective amount of the risperidone transdermal administration system of any one of claims 1-41 to a subject in need thereof.

46. A method for treating or preventing positive and negative symptoms of schizophrenia, comprising administering a therapeutically effective amount of the risperidone transdermal administration system of any one of claims 1-41 to a subject in need thereof.

47. The method of claim 46, wherein the positive and negative symptoms of schizophrenia include hallucinations, delusions, and emotional withdrawal and blunted affect.

48. The use of any one of claims 42-44 or the method of any one of claims 45-47, wherein the risperidone transdermal administration system is administered once every 1 day, every 3 days, every 7 days, every 10 days, or every 14 days.

49. The use of any one of claims 42-44 or the method of any one of claims 45-47, wherein the risperidone transdermal administration system continuously delivers risperidone or the pharmaceutically acceptable salt thereof at a therapeutically effective amount of blood drug concentration within 24 hours to 14 days.

50. The use of any one of claims 42-44 or the method of any one of claims 45-47, wherein the risperidone transdermal administration system continuously delivers risperidone into a patient at a substantially constant rate within 24 hours to 14 days, preferably within 24 hours to 7 days.

51. The use or method of claim 50, wherein the maximum transdermal amount of risperidone occurs within 24-36 hours after administration of the risperidone transdermal administration system, and during day 3-day 7 after the administration, the transdermal amount of risperidone is maintained more than 65% of the maximum transdermal amount, preferably, maintained 65%-90% of the maximum transdermal amount of risperidone, more preferably, maintained 75%-85% of the maximum transdermal amount of risperidone.
